(19) United States
(12) Patent Application Publication
(10) Pub. No.: US 2014/0058261 A1
(43) Pub. Date: Feb. 27, 2014

(54) ULTRASONIC DIAGNOSTIC APPARATUS

(71) Applicants: Toshiba Medical Systems Corporation, Otawara-shi (JP); Kabushiki Kaisha Toshiba, Tokyo (JP)

(72) Inventors: Kenichi Ichioka, Nasushiobara-shi (JP); Shigemitsu Nakaya, Nasushiobara-shi (JP); Tomohisa Imamura, Nasushiobara-shi (JP)

(73) Assignees: Toshiba Medical Systems Corporation, Otawara-shi (JP); Kabushiki Kaisha Toshiba, Tokyo (JP)

(21) Appl. No.: 14/065,927

(22) Filed: Oct. 29, 2013

Related U.S. Application Data
(63) Continuation of application No. PCT/JP2012/062664, filed on May 17, 2012.

(30) Foreign Application Priority Data
May 26, 2011 (JP) 2011-118328

(51) Int. Cl. A61B 8/00; A61B 8/12; A61B 8/08

(52) U.S. Cl. CPC A61B 8/466; A61B 8/463; A61B 8/5207; A61B 8/4263; A61B 8/54; A61B 8/4444; A61B 8/12
USPC 600/440

(57) ABSTRACT

An ultrasonic diagnostic apparatus according to an embodiment includes a display unit, an image generator, an acquiring unit, a rendering processor, and a controller. The display unit displays a stereoscopic image by displaying a parallax image group. The image generator generates an ultrasound image based on reflection waves received by an ultrasound probe held against a subject. The acquiring unit acquires three-dimensional position information of the ultrasound probe of when an ultrasound image is captured. The rendering processor generates a probe image group for allowing the ultrasound probe to be virtually perceived as a stereoscopic image based on the three-dimensional position information. The controller controls to display a characterizing image depicting a characteristic of a condition under which the ultrasound image is captured and the probe image group onto the display unit in a positional relationship based on the three-dimensional position information.

### CROSS-REFERENCE TO RELATED APPLICATIONS

This application is a continuation of PCT international application Ser. No. PCT/JP2012/062664 filed on May 17, 2012 which designates the United States, incorporated herein by reference, and which claims the benefit of priority from Japanese Patent Application No. 2011-118328, filed on May 26, 2011, the entire contents of which are incorporated herein by reference.

### FIELD

Embodiments described herein relate generally to an ultrasonic diagnostic apparatus.

### BACKGROUND

Ultrasonic diagnostic apparatuses play an important role in today's medical care, because ultrasonic diagnostic apparatuses are capable of generating and displaying an ultrasound image representing tissues directly below where an ultrasound probe is held against, in real-time.

In addition, known is a technology for automatically displaying a “mark” indicating information of a region where an image is captured onto a monitor, to contribute to information provisioning to radiologists or reproducibility in re-examinations. A “mark” herein is a mark indicating an organ to be examined (referred to as a body mark or a pictogram), or a mark indicating where in the organ is scanned with an ultrasonic wave (referred to as a probe mark).

By looking at the probe mark plotted on the body mark displayed with an ultrasound image on the monitor, an observer (a radiologist or an ultrasonographer) can read position information of the ultrasound probe and the scanned direction. However, information that can be read from these “marks” displayed on the monitor is two-dimensional information. Therefore, an observer of the monitor cannot read three-dimensional information related to the operation of the ultrasound probe performed on the body surface of a subject by an operator, such as an ultrasonographer, in order to capture the ultrasound image suitable for interpretations.

### BRIEF DESCRIPTION OF THE DRAWINGS

FIG. 1 is a schematic for explaining an exemplary structure of an ultrasonic diagnostic apparatus according to a first embodiment;

FIG. 2A and FIG. 2B are schematics for explaining an example of a stereoscopic display monitor providing a stereoscopic vision using a two-parallax image;

FIG. 3 is a schematic for explaining an example of a stereoscopic display monitor providing a stereoscopic vision using a nine-parallax image;

FIG. 4 is a schematic for explaining an example of a volume rendering process for generating a parallax image group;

FIG. 5A, FIG. 5B, FIG. 6, FIG. 7A, FIG. 7B, and FIG. 7C are schematics for explaining the acquiring device;

FIG. 8 and FIG. 9 are schematics for explaining the example of the display control performed by the controller according to the first embodiment;

FIG. 10 and FIG. 11 are schematics for explaining another mode of the display control performed by the controller according to the first embodiment explained with reference to FIGS. 8 and 9;

FIG. 12 is a flowchart for explaining a process performed by the ultrasonic diagnostic apparatus according to the first embodiment;

FIG. 13 is a schematic for explaining a variation of how the three-dimensional position information is acquired;

FIG. 14 is a schematic for explaining a second embodiment;

FIG. 15 is a flowchart for explaining a process performed by an ultrasonic diagnostic apparatus according to the second embodiment;

FIG. 16A, FIG. 16B, and FIG. 17 are schematics for explaining the third embodiment;

FIG. 18 is a flowchart for explaining a process performed by an ultrasonic diagnostic apparatus according to the third embodiment; and

FIG. 19 and FIG. 20 are schematics for explaining the variation of the first to the third embodiments.

### DETAILED DESCRIPTION

An ultrasonic diagnostic apparatus according to an embodiment includes a display unit, an image generator, an acquiring unit, a rendering processor, and a controller. The display unit is configured to display a stereoscopic image that is stereoscopically perceived by an observer, by displaying a parallax image group that is parallax images having a given parallax number. The image generator is configured to generate an ultrasound image based on reflection waves received by an ultrasound probe held against a subject. The acquiring unit is configured to acquire three-dimensional position information of the ultrasound probe of when an ultrasound image is captured. The rendering processor is configured to generate a probe image group that is a parallax image group for allowing the ultrasound probe to be virtually perceived as a stereoscopic image through a volume rendering process based on the three-dimensional position information acquired by the acquiring unit. The controller configured to control to display at least one of the ultrasound image and an abutting surface image depicting an abutting surface of the subject against which the ultrasound probe is held, as a characterizing image depicting a characteristic of a condition under which the ultrasound image is captured, and the probe image group onto the display unit in a positional relationship based on the three-dimensional position information.

An ultrasonic diagnostic apparatus according to an embodiment will be explained in detail with reference to the accompanying drawings.

To begin with, terms used in the embodiment below will be explained. A “parallax image group” is a group of images generated by applying a volume rendering process to volume data while shifting viewpoint positions by a given parallax angle. In other words, a “parallax image group” includes a plurality of “parallax images” each of which has a different “viewpoint position”. A “parallax angle” is an angle determined by adjacent viewpoint positions among viewpoint positions specified for generation of the “parallax image group” and a given position in a space represented by the volume data (e.g., the center of the space). A “parallax number” is the number of “parallax images” required for a stereoscopic vision on a stereoscopic display monitor. A “nine-parallax image” mentioned below means a “parallax image group” with nine “parallax images”. A “two-parallax image” mentioned below means a “parallax image group” with two “parallax images”. A “stereoscopic image” is an image stereoscopically perceived by an observer who is looking at a stereoscopic display monitor displaying a parallax image group.

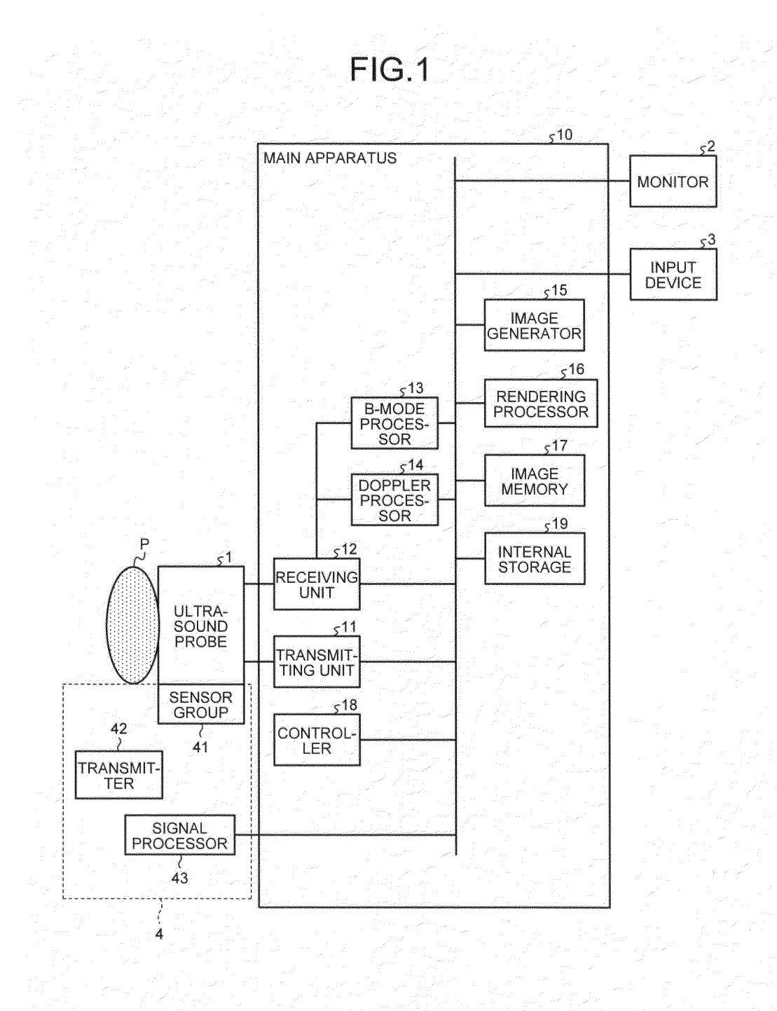

A structure of an ultrasonic diagnostic apparatus according to a first embodiment will be explained. FIG. 1 is a schematic for explaining an example of an exemplary structure of an ultrasonic diagnostic apparatus according to the first embodiment. As illustrated in FIG. 1, the ultrasonic diagnostic apparatus according to the first embodiment includes an ultrasound probe 1, a monitor 2, an input device 3, an acquiring device 4, and a main apparatus 10.

The ultrasound probe 1 includes a plurality of piezoelectric transducer elements. The piezoelectric transducer elements generate ultrasonic waves based on driving signals supplied by a transmitting unit 11 provided in the main apparatus 10, which is to be explained later. The ultrasound probe 1 also receives reflection waves from a subject P and converts the reflection waves into electrical signals. The ultrasound probe 1 also includes matching layers provided on the piezoelectric transducer elements, and a backing material for preventing the ultrasonic waves from propagating backwardly from the piezoelectric transducer elements. The ultrasound probe 1 is connected to the main apparatus 10 in a removable manner.

When an ultrasonic wave is transmitted from the ultrasound probe 1 toward the subject P, the ultrasonic wave thus transmitted is reflected one after another on a discontinuous acoustic impedance surface in body tissues within the subject P, and received as reflection wave signals by the piezoelectric transducer elements in the ultrasonic probe 1. The amplitude of the reflection wave signals thus received depends on an acoustic impedance difference on the discontinuous surface on which the ultrasonic wave is reflected. When a transmitted ultrasonic wave pulse is reflected on a moving blood flow or the surface of a cardiac wall, the frequency of the reflection wave signal thus received is shifted by the Doppler shift depending on the velocity component of the moving object with respect to the direction in which the ultrasonic wave is transmitted.

The first embodiment is applicable to both cases where the ultrasound probe 1 is an ultrasound probe that scans the subject P two-dimensionally with an ultrasonic wave, and where the ultrasound probe 1 is an ultrasound probe that scans the subject P three-dimensionally. Known as the ultrasound probe 1 that scans the subject P three-dimensionally is a mechanical scanning probe that scans the subject P three-dimensionally by swinging a plurality of ultrasound transducer elements scanning the subject P two-dimensionally by a predetermined angle (swinging angle). Known as the ultrasound probe 1 that scans the subject P three-dimensionally is a two-dimensional ultrasound probe that is capable of performing three-dimensional ultrasound scanning on the subject P with a plurality of ultrasound transducer elements arranged in a matrix. Such a two-dimensional ultrasound probe is also capable of scanning the subject P two-dimensionally by converging the ultrasonic wave and transmitting the converged ultrasonic wave.

Explained below is an example in which the ultrasound probe 1 is an ultrasound probe scanning the subject P two-dimensionally with an ultrasonic wave.

The input device 3 includes a mouse, a keyboard, a button, a panel switch, a touch command screen, a foot switch, a track ball, a joystick, and a haptic device, for example. The input device 3 receives various setting requests from an operator of the ultrasonic diagnostic apparatus, and forwards the various setting requests thus received to the main apparatus 10.

The monitor 2 displays a graphical user interface (GUI) for allowing the operator of the ultrasonic diagnostic apparatus to input various setting requests using the input device 3, and an ultrasound image generated by the main apparatus 10, for example.

The monitor 2 according to the first embodiment is a monitor that displays a stereoscopic image that is stereoscopically perceived by an observer by displaying a group of parallax images in a given parallax number (hereinafter, referred to as a stereoscopic display monitor). A stereoscopic display monitor will now be explained.

A common, general-purpose monitor that is most widely used today displays two-dimensional images two-dimensionally, and is not capable of displaying a two-dimensional image stereoscopically. If an observer requests a stereoscopic vision on the general-purpose monitor, an apparatus outputting images to the general-purpose monitor needs to display two-parallax images in parallel that can be perceived by the observer stereoscopically, using a parallel technique or a crossed-eye technique. Alternatively, the apparatus outputting images to the general-purpose monitor needs to present images that can be perceived stereoscopically by the observer with anaglyph, which uses a pair of glasses having a red filter for the left eye and a blue filter for the right eye, using a complementary color method, for example.

Some stereoscopic display monitors display two-parallax images (also referred to as binocular parallax images) to enable stereoscopic vision using binocular parallax (hereinafter, also mentioned as a two-parallax monitor).

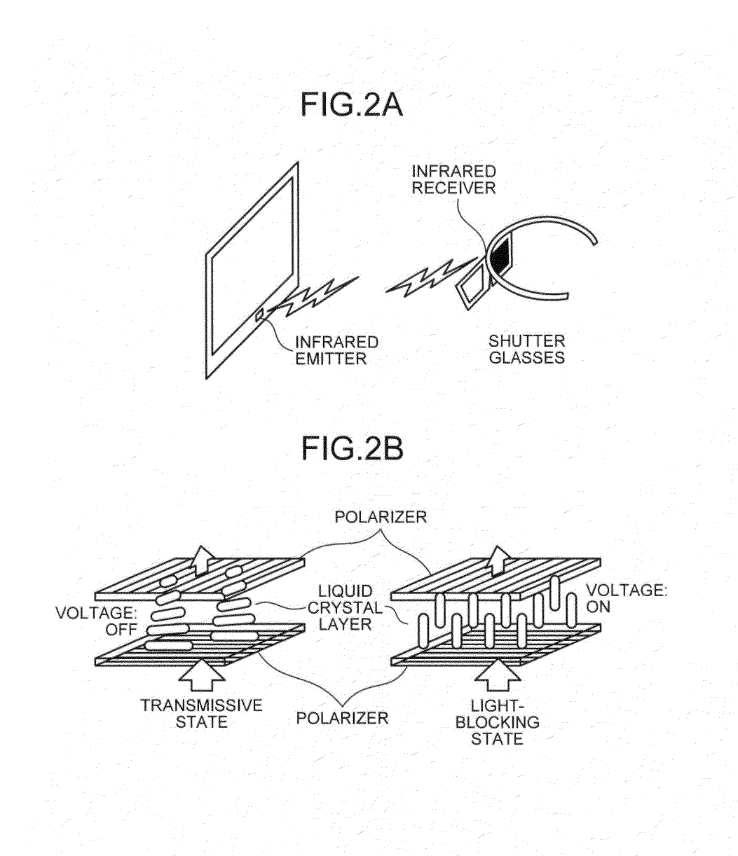

FIGS. 2A and 2B are schematics for explaining an example of a stereoscopic display monitor providing a stereoscopic vision using two-parallax images. The example illustrated in FIGS. 2A and 2B represents a stereoscopic display monitor providing a stereoscopic vision using a shutter technique. In this example, a pair of shutter glasses is used as stereoscopic glasses worn by an observer who observes the monitor. The stereoscopic display monitor outputs two-parallax images onto the monitor alternatingly. For example, the monitor illustrated in FIG. 2A outputs an image for the left eye and an image for the right eye alternatingly at 120 hertz. An infrared emitter is installed in the monitor, as illustrated in FIG. 2A, and the infrared emitter controls infrared outputs based on the timing at which the images are swapped.

The infrared output from the infrared emitter is received by an infrared receiver provided on the shutter glasses illustrated in FIG. 2A. A shutter is installed on the frame on each side of the shutter glasses. The shutter glasses switch the right shutter and the left shutter between a transmissive state and a light-blocking state alternatingly, based on the timing at which the infrared receiver receives infrared. A process of switching the shutters between the transmissive state and the light-blocking state will now be explained.

As illustrated in FIG. 2B, each of the shutters includes an incoming polarizer and an outgoing polarizer, and also includes a liquid crystal layer interposed between the incoming polarizer and the outgoing polarizer. The incoming polarizer and the outgoing polarizer are orthogonal to each other, as illustrated in FIG. 2B. In an “OFF” state during which a voltage is not applied as illustrated in FIG. 2B, the light having passed through the incoming polarizer is rotated by 90 degrees by the effect of the liquid crystal layer, and thus passes through the outgoing polarizer. In other words, a shutter with no voltage applied is in the transmissive state.

By contrast, as illustrated in FIG. 2B, in an “ON” state during which a voltage is applied, the polarization rotation effect of liquid crystal molecules in the liquid crystal layer is lost. Therefore, the light having passed through the incoming polarizer is blocked by the outgoing polarizer. In other words, the shutter applied with a voltage is in the light-blocking state.

The infrared emitter outputs infrared for a time period while which an image for the left eye is displayed on the monitor, for example. During the time the infrared receiver is receiving infrared, no voltage is applied to the shutter for the left eye, while a voltage is applied to the shutter for the right eye. In this manner, as illustrated in FIG. 2A, the shutter for the right eye is in the light-blocking state and the shutter for the left eye is in the transmissive state to cause the image for the left eye to enter the left eye of the observer. For a time period while which an image for the right eye is displayed on the monitor, the infrared emitter stops outputting infrared. When the infrared receiver receives no infrared, a voltage is applied to the shutter for the left eye, while no voltage is applied to the shutter for the right eye. In this manner, the shutter for the left eye is in the light-blocking state, and the shutter for the right eye is in the transmissive state to cause the image for the right eye to enter the right eye of the observer. As explained above, the stereoscopic display monitor illustrated in FIGS. 2A and 2B makes a display that can be stereoscopically perceived by the observer, by switching the states of the shutters in association with the images displayed on the monitor.

In addition to apparatuses providing a stereoscopic vision using the shutter technique, known as two-parallax monitors are an apparatus using a pair of polarized glasses and an apparatus using a parallax barrier and providing a stereoscopic vision.

Some stereoscopic display monitors that have recently been put into practical use allow multiple parallax images, e.g., nine-parallax images, to be stereoscopically viewed by an observer with the naked eyes, by adopting a light ray controller such as a lenticular lens. This type of stereoscopic display monitor enables stereoscopic viewing due to binocular parallax, and further enables stereoscopic viewing due to motion parallax that provides an image varying according to motion of the viewpoint of the observer.

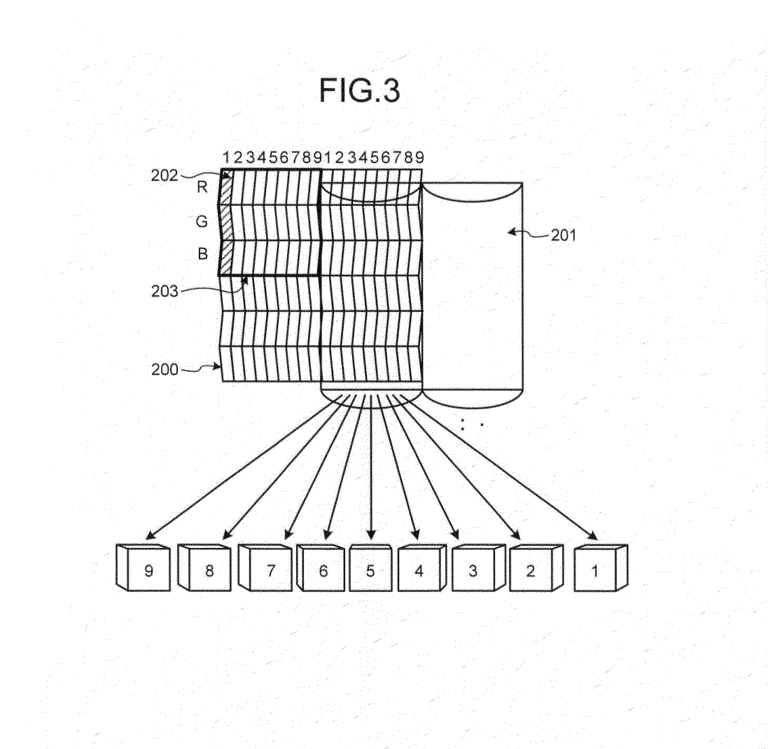

FIG. 3 is a schematic for explaining an example of a stereoscopic display monitor providing a stereoscopic vision using nine-parallax images. In the stereoscopic display monitor illustrated in FIG. 3, a light ray controller is arranged on the front surface of a flat display screen 200 such as a liquid crystal panel. For example, in the stereoscopic display monitor illustrated in FIG. 3, a vertical lenticular sheet 201 having an optical aperture extending in a vertical direction is fitted on the front surface of the display screen 200 as a light ray controller. Although the vertical lenticular sheet 201 is fitted so that the convex of the vertical lenticular sheet 201 faces the front side in the example illustrated in FIG. 3, the vertical lenticular sheet 201 may be also fitted so that the convex faces the display screen 200.

As illustrated in FIG. 3, the display screen 200 has pixels 202 that are arranged in a matrix. Each of the pixels 202 has an aspect ratio of 3:1, and includes three sub-pixels of red (R), green (G), and blue (B) that are arranged vertically. The stereoscopic display monitor illustrated in FIG. 3 converts nine-parallax images consisting of nine images into an intermediate image in a given format (e.g., a grid-like format), and outputs the result onto the display screen 200. In other words, the stereoscopic display monitor illustrated in FIG. 3 assigns and outputs nine pixels located at the same position in the nine-parallax images to the pixels 202 arranged in nine columns. The pixels 202 arranged in nine columns function as a unit pixel set 203 that displays nine images from different viewpoint positions at the same time.

The nine-parallax images simultaneously output as the unit pixel set 203 onto the display screen 200 are radiated with a light emitting diode (LED) backlight, for example, as parallel rays, and travel further in multiple directions through the vertical lenticular sheet 201. Light for each of the pixels included in the nine-parallax images is output in multiple directions, whereby the light entering the right eye and the left eye of the observer changes as the position (viewpoint position) of the observer changes. In other words, depending on the angle from which the observer perceives, the parallax image entering the right eye and the parallax image entering the left eye are at different parallax angles. Therefore, the observer can perceive a captured object stereoscopically from any one of the nine positions illustrated in FIG. 3, for example. At the position “5” illustrated in FIG. 3, the observer can perceive the captured object stereoscopically as the object faces directly the observer. At each of the positions other than the position “5” illustrated in FIG. 3, the observer can perceive the captured object stereoscopically with its orientation changed. The stereoscopic display monitor illustrated in FIG. 3 is merely an example. The stereoscopic display monitor for displaying nine-parallax images may be a liquid crystal with horizontal stripes of “RRR . . . , GGG . . . , BBB . . . ” as illustrated in FIG. 3, or a liquid crystal with vertical stripes of “RGBRGB . . . ”. The stereoscopic display monitor illustrated in FIG. 3 may be a monitor using a vertical lens in which the lenticular sheet is arranged vertically as illustrated in FIG. 3, or a monitor using a diagonal lens in which the lenticular sheet is arranged diagonally. Hereinafter, the stereoscopic display monitor explained with reference to FIG. 3 is referred to as a nine-parallax monitor.

In other words, the two-parallax monitor is a stereoscopic display monitor that displays a stereoscopic image that is perceived by an observer by displaying a parallax image group that are two-parallax image having a given parallax angle between these images (two-parallax image). The nine-parallax monitor is a stereoscopic display monitor that displays a stereoscopic image that is perceived by an observer by displaying a parallax image group that are nine-parallax images having a given parallax angle between the images (nine-parallax images).

The first embodiment is applicable to both examples in which the monitor 2 is a two-parallax monitor, and in which the monitor 2 is a nine-parallax monitor. Explained below is an example in which the monitor 2 is a nine-parallax monitor.

Referring back to FIG. 1, the acquiring device 4 acquires three-dimensional position information of the ultrasound probe 1. Specifically, the acquiring device 4 is a device that acquires three-dimensional position information of the ultrasound probe 1 of when an ultrasound image is captured. More specifically, the acquiring device 4 is a device that acquires three-dimensional position information of the ultrasound probe 1 with respect to an abutting surface of the subject P against which the ultrasound probe 1 is held when the ultrasound image is captured. If the ultrasound probe 1 is an external probe, an abutting surface would be a body surface of the subject P. In such a case, the acquiring device 4 acquires the three-dimensional position information of the ultrasound probe 1 with respect to the body surface of the subject P of when an ultrasound image is captured. When the ultrasound probe 1 is a luminal probe, such as a transesophageal echocardiographic (TEE) probe used in transesophageal echocardiography, the abutting surface would be the inner wall of the lumen in which the ultrasound probe 1 is inserted in the subject P. In such a case, the acquiring device 4 acquires three-dimensional position information of the ultrasound probe 1 with respect to the interluminal wall of the subject P of when an ultrasound image is captured. The three-dimensional position information of the ultrasound probe 1 acquired by the acquiring device 4 according to the embodiment is not limited to the three-dimensional position information of the ultrasound probe 1 with respect to the abutting surface. In the embodiment, “a sensor or a transmitter transmitting a magnetic signal” for establishing a reference position may be mounted on the ultrasonic diagnostic apparatus or a bed, for example, and a position of the ultrasound probe 1 with respect to the sensor or the transmitter thus mounted may be used as the three-dimensional position information of the ultrasound probe 1.

For example, the acquiring device 4 includes a sensor group 41 being position sensors mounted on the ultrasound probe 1, a transmitter 42, and a signal processor 43. The sensor group 41 includes position sensors, an example of which includes magnetic sensors. The transmitter 42 is arranged at any desired position, and generates a magnetic field outwardly from the acquiring device 4 as the center.

The sensor group 41 detects three-dimensional magnetic field generated by the transmitter 42, converts the magnetic field information thus detected into a signal, and outputs the signal to the signal processor 43. The signal processor 43 calculates the positions (coordinates) of the sensor group 41 within a space having a point of origin at the transmitter 42 based on the signals received from the sensor group 41, and outputs the positions thus calculated to a controller 18, which is to be described later. An image of the subject P is captured within a range of the magnetic field in which the sensor group 41 mounted on the ultrasound probe 1 is capable of detecting the magnetic field of the transmitter 42 accurately.

The sensor group 41 according to the first embodiment will be explained later in detail.

The main apparatus 10 illustrated in FIG. 1 is an apparatus that generates ultrasound image data based on reflection waves received by the ultrasound probe 1. The main apparatus 10 includes a transmitting unit 11, a receiving unit 12, a B-mode processor 13, a Doppler processor 14, an image generator 15, a rendering processor 16, an image memory 17, a controller 18, and an internal storage 19.

The transmitting unit 11 includes a trigger generator circuit, a transmission delay circuit, a pulser circuit, and the like, and supplies a driving signal to the ultrasound probe 1. The pulser circuit generates a rate pulse used in generating ultrasonic waves to be transmitted, repeatedly at a given rate frequency. The transmission delay circuit adds a delay time corresponding to each of the piezoelectric transducer elements to each of the rate pulses generated by the pulser circuit. Such a delay time is required for determining transmission directivity by converging the ultrasonic waves generated by the ultrasound probe 1 into a beam. The trigger generator circuit applies a driving signal (driving pulse) to the ultrasound probe 1 at the timing of the rate pulse. In other words, by causing the delay circuit to change the delay time to be added to each of the rate pulses, the direction in which the ultrasonic wave is transmitted from a surface of the piezoelectric transducer element is arbitrarily adjusted.

The transmitting unit 11 has a function of changing a transmission frequency, a transmission driving voltage, and the like instantaneously before executing a certain scan sequence, based on an instruction of the controller 18 to be described later. In particular, a change in the transmission driving voltage is performed by a linear amplifier type transmission circuit that is cable of switching its values instantaneously, or a mechanism for electrically switching a plurality of power units.

The receiving unit 12 includes an amplifier circuit, an analog-to-digital (A/D) converter, an adder, and the like. The receiving unit 12 generates reflection wave data by applying various processes to the reflection wave signals received by the ultrasound probe 1. The amplifier circuit amplifies the reflection wave signal on each channel, and performs a gain correction. The A/D converter performs an A/D conversion to the reflection wave signal having gain corrected, and adds a delay time required for determining reception directivity to the digital data. The adder performs an addition to the reflection wave signals processed by the A/D converter, to generate the reflection wave data. Through the addition performed by the adder, a reflection component in the direction corresponding to the reception directivity of the reflection wave signals is emphasized.

In the manner described above, the transmitting unit 11 and the receiving unit 12 control the transmission directivity and the reception directivity of the ultrasonic wave transmissions and receptions, respectively.

When the ultrasound probe 1 is a probe capable of three-dimensional scanning, the transmitting unit 11 is also capable of transmitting a three-dimensional ultrasound beam from the ultrasound probe 1 to the subject P, and the receiving unit 12 is also capable of generating three-dimensional reflection wave data from the three-dimensional reflection wave signals received by the ultrasound probe 1.

The B-mode processor 13 receives the reflection wave data from the receiving unit 12, and performs a logarithmic amplification, an envelope detection, and the like, to generate data (B-mode data) in which signal intensity is represented as a luminance level.

The Doppler processor 14 analyzes the frequencies in velocity information included in the reflection wave data received from the receiving unit 12, and extracts blood flow, tissue, and contrast agent echo components resulted from the Doppler shift, and generates data (Doppler data) that is moving object information such as an average velocity, a variance, a power, and the like extracted for a plurality of points.

The B-mode processor 13 and the Doppler processor 14 according to the first embodiment are capable of processing both of two-dimensional reflection wave data and three-dimensional reflection wave data. In other words, the B-mode processor 13 is capable of generating three-dimensional B-mode data from three-dimensional reflection wave data, as well as generating two-dimensional B-mode data from two-dimensional reflection wave data. The Doppler processor 14 is capable of generating two-dimensional Doppler data from two-dimensional reflection wave data, and generating three-dimensional Doppler data from three-dimensional reflection wave data.

The image generator 15 generates an ultrasound image based on the reflection waves received by the ultrasound probe 1 held against the body surface of the subject P. In other words, the image generator 15 generates ultrasound image data from the data generated by the B-mode processor 13 and by the Doppler processor 14. Specifically, the image generator 15 generates B-mode image data in which the intensity of a reflection wave is represented as a luminance from two-dimensional B-mode data generated by the B-mode processor 13. The image generator 15 generates an average velocity image, a variance image, or a power image representing the moving object information, or color Doppler image data being a combination of these images, from the two-dimensional Doppler data generated by the Doppler processor 14.

Generally, the image generator 15 converts rows of scan line signals from an ultrasound scan into rows of scan line signals in a video format, typically one used for television (performs a scan conversion), to generate ultrasound image data to be displayed. Specifically, the image generator 15 generates ultrasound image data to be displayed by performing a coordinate conversion in accordance with a way in which an ultrasound scan is performed with the ultrasound probe 1. The image generator 15 also synthesizes various character information for various parameters, scales, body marks, and the like to the ultrasound image data.

The image generator 15 is also capable of generating three-dimensional ultrasound image data. In other words, the image generator 15 can generate three-dimensional B-mode image data by performing a coordinate conversion to the three-dimensional B-mode data generated by the B-mode processor 13. The image generator 15 can also generate three-dimensional color Doppler image data by performing a coordinate conversion to the three-dimensional Doppler data generated by the Doppler processor 14.

The rendering processor 16 performs various rendering processes to volume data. Specifically, the rendering processor 16 is a processor that performs various processes to volume data. Volume data is three-dimensional ultrasound image data generated by capturing images of the subject P in the real space, or virtual volume data plotted in a virtual space. For example, the rendering processor 16 performs rendering processes to three-dimensional ultrasound image data to generate two-dimensional ultrasound image data to be displayed. The rendering processor 16 also performs rendering processes to virtual volume data to generate two-dimensional image data that is to be superimposed over the two-dimensional ultrasound image data to be displayed.

The rendering processes performed by the rendering processor 16 include a process of reconstructing a multi-planer reconstruction (MPR) image by performing a multi-planer reconstruction. The rendering processes performed by the rendering processor 16 include a process of applying a “curved MPR” to the volume data, and a process of applying “intensity projection” to the volume data.

The rendering processes performed by the rendering processor 16 also include volume rendering process for generating a two-dimensional image reflected with three-dimensional information. In other words, the rendering processor 16 generates a parallax image group by performing volume rendering processes to three-dimensional ultrasound image data or virtual volume data from a plurality of viewpoint positions having the center at a reference viewpoint position. Specifically, because the monitor 2 is a nine-parallax monitor, the rendering processor 16 generates nine-parallax images by performing volume rendering processes to the volume data from nine viewpoint positions having the center at the reference viewpoint position.

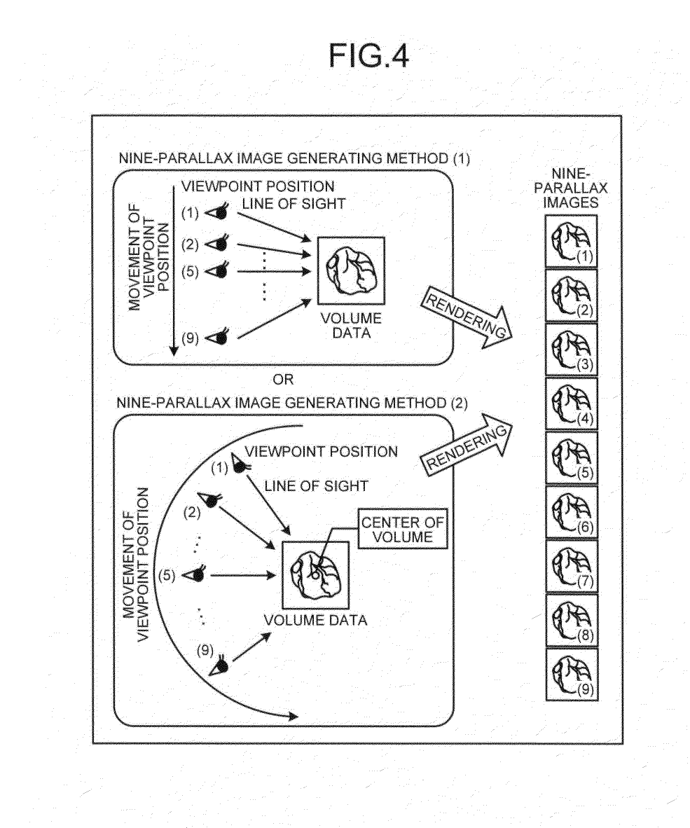

The rendering processor 16 generates nine-parallax images by performing a volume rendering process illustrated in FIG. 4 under the control of the controller 18, which is to be described later. FIG. 4 is a schematic for explaining an example of a volume rendering process for generating a parallax image group.

For example, it is assumed herein that the rendering processor 16 receives parallel projection as a rendering condition, and a reference viewpoint position (5) and a parallax angle of “one degree”, as illustrated in a “nine-parallax image generating method (1)” in FIG. 4. In such a case, the rendering processor 16 generates nine-parallax images, each having a parallax angle (angle between the lines of sight) shifted by one degree, by parallel projection, by moving a viewpoint position in parallel from (1) to (9) in such a way that the parallax angle is set in every “one degree”. Before performing parallel projection, the rendering processor 16 establishes a light source radiating parallel light rays from the infinity along the line of sight.

Alternatively, it is assumed that the rendering processor 16 receives perspective projection as a rendering condition, and a reference viewpoint position (5) and a parallax angle of “one degree”, as illustrated in “nine-parallax image generating method (2)” in FIG. 4. In such a case, the rendering processor 16 generates nine-parallax images, each having a parallax angle shifted by one degree, by perspective projection, by moving the viewpoint position from (1) to (9) around the center (the center of gravity) of the volume data in such a way that the parallax angle is set in every “one degree”. Before performing perspective projection, the rendering processor 16 establishes a point light source or a surface light source radiating light three-dimensionally about the line of sight, for each of the viewpoint positions. Alternatively, when perspective projection is to be performed, the viewpoint position (1) to (9) may be shifted in parallel depending on rendering conditions.

The rendering processor 16 may also perform a volume rendering process using both parallel projection and perspective projection, by establishing a light source radiating light two-dimensionally, radially from a center on the line of sight for the vertical direction of the volume rendering image to be displayed, and radiating parallel light rays from the infinity along the line of sight for the horizontal direction of the volume rendering image to be displayed.

The nine-parallax images thus generated correspond to a parallax image group. In other words, the parallax image group is a group of images for a stereoscopic vision, generated from the volume data.

When the monitor 2 is a two-parallax monitor, the rendering processor 16 generates two-parallax images by setting two viewpoint positions, for example, having a parallax angle of “one degree” from the center at the reference viewpoint position.

The rendering processor 16 also has a drawing function for generating a two-dimensional image in which a given form is represented.

As mentioned earlier, the rendering processor 16 generates a parallax image group through the volume rendering process, not only from three-dimensional ultrasound image data but also from virtual volume data. The image generator 15 generates a synthesized image group in which ultrasound image data and a parallax image group generated by the rendering processor 16 are synthesized. The parallax image group generated from the virtual volume data by the rendering processor 16 according to the first embodiment and the synthesized image group generated by the image generator 15 according to the first embodiment will be explained later in detail.

The image memory 17 is a memory for storing therein image data generated by the image generator 15 and the rendering processor 16. The image memory 17 can also store therein data generated by the B-mode processor 13 and the Doppler processor 14.

The internal storage 19 stores therein control programs for transmitting and receiving ultrasonic waves, performing image processes and displaying processes, and various data such as diagnostic information (e.g., a patient identification (ID) and observations by a doctor), a diagnostic protocol, and various body marks, and the like. The internal storage 19 is also used for storing therein the image data stored in the image memory 17, for example, as required.

The internal storage 19 also stores therein offset information for allowing the acquiring device 4 to acquire the position information of the sensor group 41 with respect to an abutting surface (e.g., body surface) of the subject P as three-dimensional position information of the ultrasound probe 1. The offset information will be described later in detail.

The controller 18 controls the entire process performed by the ultrasonic diagnostic apparatus. Specifically, the controller 18 controls the processes performed by the transmitting unit 11, the receiving unit 12, the B-mode processor 13, the Doppler processor 14, the image generator 15, and the rendering processor 16 based on various setting requests input by the operator via the input device 3, or various control programs and various data read from the internal storage 19. For example, the controller 18 controls the volume rendering process performed by the rendering processor 16 based on the three-dimensional position information of the ultrasound probe 1 acquired by the acquiring device 4.

The controller 18 also controls to display ultrasound image data to be displayed stored in the image memory 17 or the internal storage 19 onto the monitor 2. Specifically, the controller 18 according to the first embodiment displays a stereoscopic image that can be perceived stereoscopically by an observer (an operator of the ultrasonic diagnostic apparatus) by converting the nine-parallax images into an intermediate image in which the parallax image group is arranged in a predetermined format (e.g., a grid-like format), and outputting the intermediate image to the monitor 2 being a stereoscopic display monitor.

The overall structure of the ultrasonic diagnostic apparatus according to the first embodiment is explained above. The ultrasonic diagnostic apparatus according to the first embodiment having such a structure performs a process described below to provide three-dimensional information related to an operation of the ultrasound probe 1.

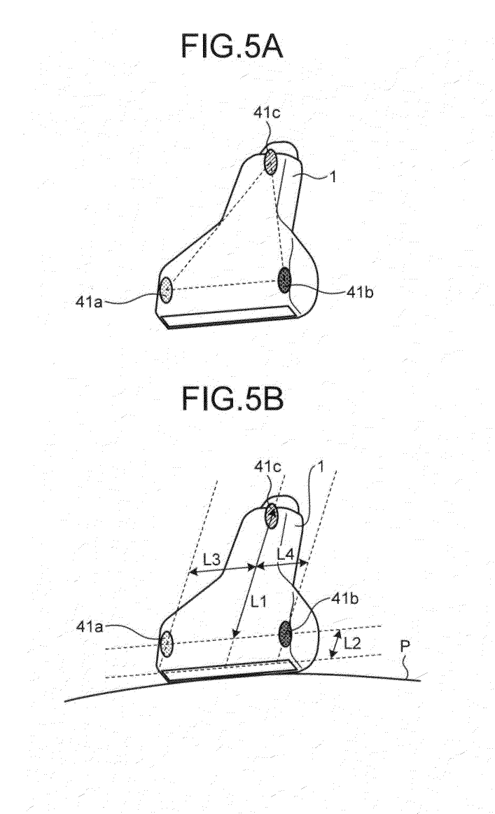

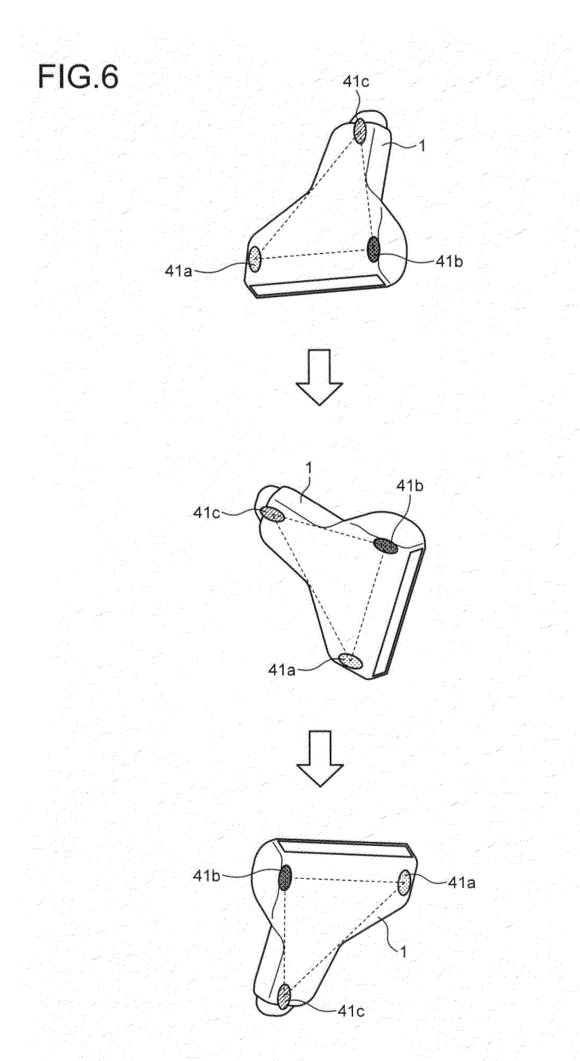

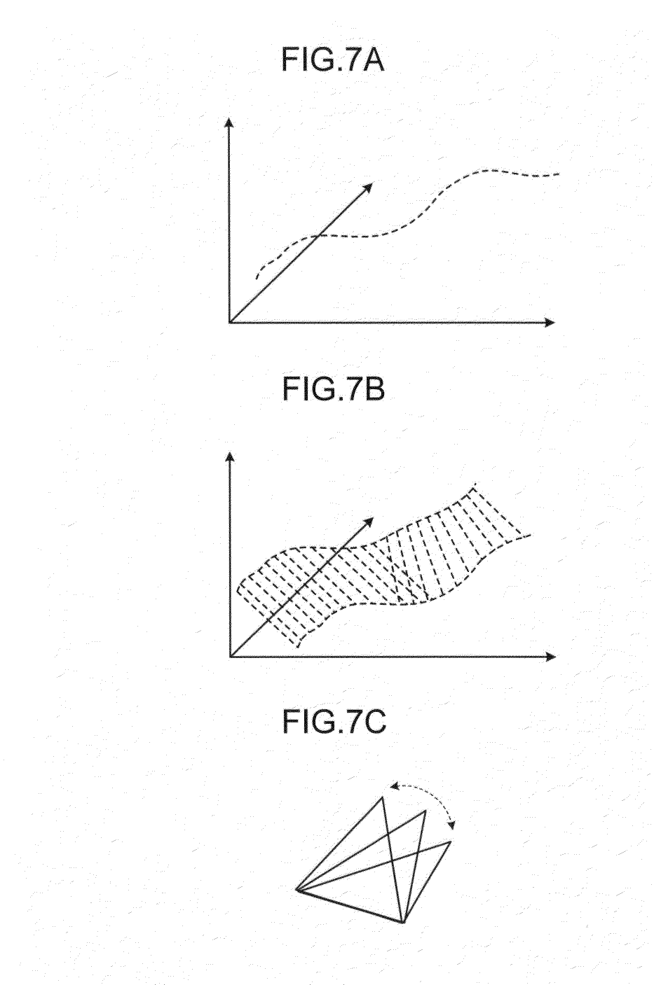

As mentioned above, the acquiring device 4 acquires the three-dimensional position information of the ultrasound probe 1 of when an ultrasound image is captured. Specifically, the acquiring device 4 acquires the three-dimensional position information using the position sensors (the sensor group 41) mounted on the ultrasound probe 1. FIGS. 5A, 5B, 5C, 6, 7A, 7B, and 7C are schematics for explaining the acquiring device.

For example, as illustrated in FIG. 5A, three magnetic sensors that are a magnetic sensor 41a, a magnetic sensor 41b, and a magnetic sensor 41c are mounted on the surface of the ultrasound probe 1 as the sensor group 41. The magnetic sensor 41a and the magnetic sensor 41b are mounted in parallel with a direction in which the transducer elements are arranged, as illustrated in FIG. 5A. The magnetic sensor 41c is mounted near the top end of the ultrasound probe 1, as illustrated in FIG. 5A.

As information of positions where the sensor group 41 is mounted, for example, offset information (L1 to L4) illustrated in FIG. 5B is stored in the internal storage 19. The distance “L1” is a distance between a line connecting positions where the magnetic sensor 41a and the magnetic sensor 41b are mounted and a position where the magnetic sensor 41c is mounted, as illustrated in FIG. 5B.

The distance “L2” is a distance between the line connecting the positions where the magnetic sensor 41a and the magnetic sensor 41b are mounted and the surface on which the transducer elements are arranged. In other words, the distance “L2” represents a distance between the line connecting the positions where the magnetic sensor 41a and the magnetic sensor 41b are mounted and the abutting surface (for example, body surface of the subject P), as illustrated in FIG. 5B.

The distance “L3” is a distance between the magnetic sensor 41a and the magnetic sensor 41c along the direction in which the transducer elements are arranged, as illustrated in FIG. 5B. The distance “L4” is a distance between the magnetic sensor 41b and the magnetic sensor 41c in the direction in which the transducer elements are arranged, as illustrated in FIG. 5B.

To capture a B-mode image most suitable for image diagnosis, for example, an operator moves the ultrasound probe 1 to different directions while holding the ultrasound probe 1 against the body surface of the subject P, as illustrated in FIG. 6. The signal processor 43 in the acquiring device 4 can acquire three-dimensional position information of the ultrasound probe 1 with respect to the body surface of the subject P of when the image is captured, using the offset information illustrated in FIG. 5B, from acquired positions (coordinates) of the sensor group 41, as illustrated in FIG. 6.

Before causing the acquiring device 4 to acquire the three-dimensional position information of the ultrasound probe 1, an operator may choose a pattern for acquiring the three-dimensional position information, as required. For example, when an operator captures an image by moving the ultrasound probe 1 in parallel, while keeping the angle of the ultrasound probe 1 with respect to the subject P fixed, the operator chooses causing the acquiring device 4 to acquire the position of only one of the magnetic sensors in the sensor group 41 or the position of the gravity center of the sensor group 41 (first acquiring pattern). When the first acquiring pattern is selected, the acquiring device 4 acquires the three-dimensional position information of the ultrasound probe 1 in the real space as a single trajectory, as illustrated in FIG. 7A. The three-dimensional position information illustrated in FIG. 7A represents information of a position on a body surface or an interluminal wall of the subject P against which the ultrasound probe 1 is held in contact. When the reference position is set to a bed or to the main unit of the ultrasonic diagnostic apparatus, three-dimensional position information is represented as information of a position in absolute coordinates, instead of as a relationship with respect to the subject P. The first acquiring pattern is selected, for example, when ultrasound elastography, in which an operator moves the ultrasound probe 1 up and down in the vertical directions with respect to a body surface, is conducted.

There are also situations where an operator captures an image by moving the ultrasound probe 1 in different directions while keeping the angle of the ultrasound probe 1 with respect to the subject P fixed, for example. In such a case, the operator selects to cause the acquiring device 4 to acquire the position of the magnetic sensor 41a and the magnetic sensor 41b (second acquiring pattern), for example. When the second acquiring pattern is selected, the acquiring device 4 acquires the three-dimensional position information of the ultrasound probe 1 in the real space as two trajectories, as illustrated in FIG. 7B. The three-dimensional position information illustrated in FIG. 7B represents a position on a body surface or an interluminal wall of the subject P against which the ultrasound probe 1 is held in contact and information of a position of the ultrasound beam in a lateral direction. When the second acquiring pattern is selected, the acquiring device 4 can also acquire the three-dimensional position information of a rotating movement of the ultrasound probe 1 performed by the operator.

There are also situations where the operator captures an image by moving the ultrasound probe 1 in different angles and different directions. In such a case, the operator selects to cause the acquiring device 4 to acquire all of the positions of the sensor group 41 (third acquiring pattern). When the third acquiring pattern is selected, the acquiring device 4 acquires the three-dimensional position information of the ultrasound probe 1 in the real space as three trajectories. In this manner, the acquiring device 4 can also acquire three-dimensional position information related to a degree by which the ultrasound probe 1 is inclined by the operator, as illustrated in FIG. 7B. When the third acquiring pattern is selected, the acquiring device 4 represents a position on a body surface or an interluminal wall of the subject P against which the ultrasound probe 1 is held in contact, and position information of the ultrasound beam in the lateral direction and in a depth direction. The third acquiring pattern is a pattern that is selected in a general image capturing, and selected when an apical four-chamber view is captured based on an apical approach, for example.

Explained below is an example in which a B-mode image is captured after the third acquiring pattern is selected. Explained below is an example in which an ultrasound scanning is performed with the ultrasound probe 1 that is an external probe. In other words, explained below is an example in which the abutting surface is a body surface of the subject P. In such a case, the acquiring device 4 acquires three-dimensional position information of the ultrasound probe 1 moved on the body surface of the subject P by an operation of an operator. The acquiring device 4 then notifies the controller 18 of the three-dimensional position information thus acquired. The controller 18 acquires the three-dimensional position information of the ultrasound probe 1 with respect to the body surface of when the image is captured from the acquiring device 4, and controls to perform a rendering process to virtual volume data of the ultrasound probe 1 based on the three-dimensional position information thus acquired.

Specifically, the rendering processor 16 generates a probe image group that is a parallax image group for allowing the ultrasound probe 1 to be virtually perceived as a stereoscopic image through a volume rendering process, based on the three-dimensional position information acquired by the acquiring device 4. To explain using an example, the rendering processor 16 moves virtual volume data of the ultrasound probe 1 plotted in a virtual space (hereinafter, mentioned as virtual probe three-dimensional (3D) data), in parallel or rotates the virtual volume data, based on the three-dimensional position information. The rendering processor 16 then establishes a reference viewpoint position with respect to the virtual probe 3D data thus moved. For example, the reference viewpoint position is set to a position facing directly to the captured B-mode image. The rendering processor 16 then sets up nine viewpoint positions each having a parallax angle of one degree from each other, from the reference viewpoint position located at the center, toward the center of gravity of the virtual probe 3D data, for example.

The rendering processor 16 then generates a probe image group “probe images (1) to (9)” by performing a volume rendering process using perspective projection, from the nine viewpoint positions toward the center of gravity of virtual probe 3D data, for example.

The controller 18 controls to display “at least one of an ultrasound image generated by the image generator 15 and an abutting surface image depicting the abutting surface of the subject P against which the ultrasound probe 1 is held in contact, as a characterizing image depicting a characteristic of a condition under which the image is captured” and the probe image group onto the monitor 2, in a positional relationship based on the three-dimensional position information. In the embodiment, the abutting surface image is a body surface image indicating a body surface of the subject P. For example, an ultrasound image as a characterizing image is a B-mode image generated from the reflection waves received by the ultrasound probe 1 when the acquiring device 4 acquired the three-dimensional position information used for generating the probe image group. A body surface image that is an abutting surface image as a characterizing image is, specifically, a body mark schematically depicting a region from which an ultrasound image is captured. More specifically, the body surface image as a characterizing image is a 3D body mark that is a three-dimensional representation of the captured region, or a rendering image generated from the volume data of the captured region. An example of a rendering image as a body surface image includes a surface rendering image of mammary gland tissues being a captured region. Another example of a rendering image as a body surface image includes an MPR image of mammary gland tissues being a captured region. Another example of a rendering image as a body surface image includes an image in which a surface rendering image of mammary gland tissues being a captured region is synthesized with an MPR image that is a sectional view of the mammary gland tissues.

To control to display in the manner explained above, the controller 18 causes the image generator 15 to generate a synthesized image group “synthesized images (1) to (9)”. In each one of these “synthesized images (1) to (9)”, each one of the “probe images (1) to (9)” is synthesized with a B-mode image in a positional relationship based on the three-dimensional position information, for example. Alternatively, the controller 18 causes the image generator 15 to generate a synthesized image group “synthesized images (1) to (9)” in which each one of the “probe images (1) to (9)” is synthesized with a B-mode image and a body surface image (a 3D body mark of a breast or a rendering image of mammary gland tissues) in a positional relationship based on the three-dimensional position information, for example. The controller 18 then causes the monitor 2 to display a stereoscopic image of the synthesized image group, by causing to display the synthesized image group “synthesized images (1) to (9)”, respectively, onto the pixels 202 arranged in nine columns (see FIG. 3). The controller 18 also stores the synthesized image group (the probe image group and the characterizing image) displayed onto the monitor 2 in the image memory 17 or in the internal storage 19. For example, the controller 18 stores the synthesized image group displayed onto the monitor 2 in association with an examination ID.

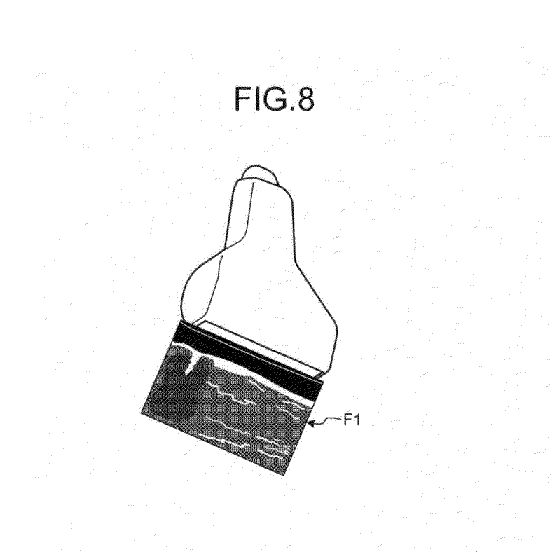

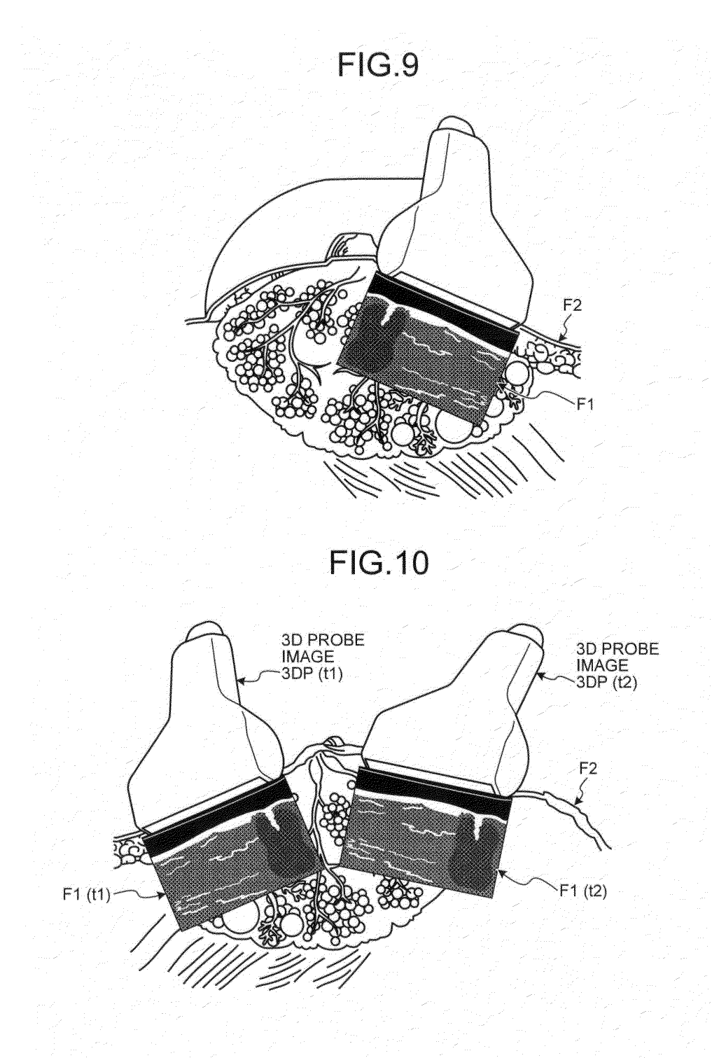

FIGS. 8 and 9 are schematics for explaining an example of the display control performed by the controller according to the first embodiment. For example, when the B-mode image is specified as a characterizing image, the monitor 2 displays a synthesized image group in which the probe image group and a B-mode image “F1” are synthesized in a positional relationship based on the three-dimensional position information, under the display control performed by the controller 18, as illustrated in FIG. 8.

When the B-mode image and the rendering image of a captured region are specified as a characterizing image, for example, the monitor 2 displays a synthesized image group in which the probe image group, the B-mode image “F1”, and a rendering image “F2” of mammary gland tissues are synthesized in a positional relationship based on the three-dimensional position information, under the display control performed by the controller 18, as illustrated in FIG. 9.

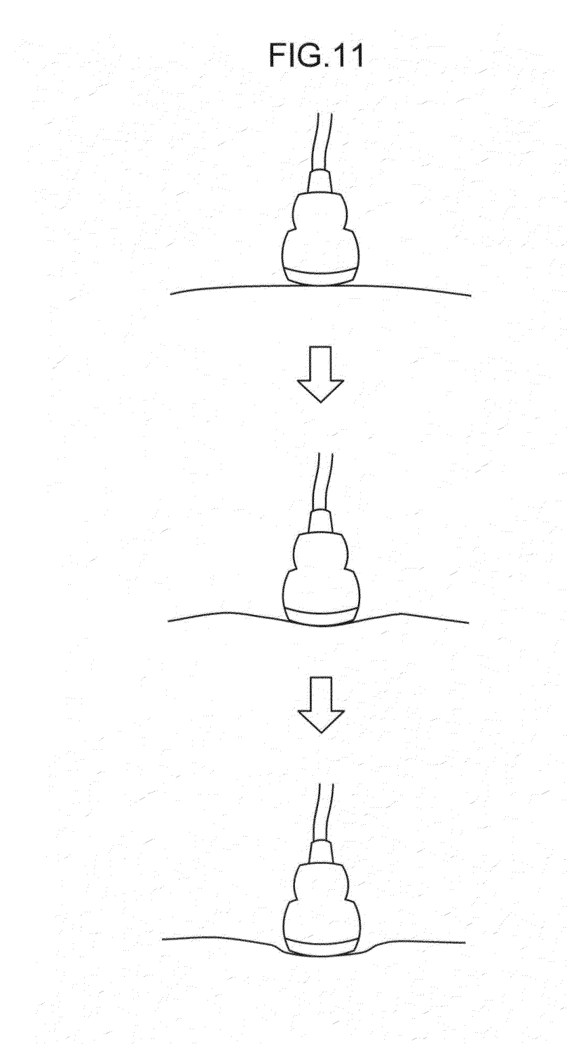

Displayed and stored in the example explained above is a synthesized image group of when a specific ultrasound image is captured. However, the embodiment is also applicable to an example in which a plurality of synthesized image groups of when a plurality of ultrasound images is captured are displayed and stored. FIGS. 10 and 11 are schematics for explaining another example of the display control performed by the controller according to the first embodiment explained with reference to FIGS. 8 and 9.

The image generator 15 generates a plurality of ultrasound images in a temporal order, based on reflection waves received by the ultrasound probe 1 in the temporal order. Specifically, while an operator is operating the ultrasound probe 1 to capture a B-mode image in which a tumor region of a breast is clearly represented, the image generator 15 generates a plurality of B-mode images in the temporal order. For example, the image generator 15 generates a B-mode image “F1(t1)” at time “t1”, and a B-mode image “F1(t2)” at time “t2”.

The acquiring device 4 acquires temporal-order three-dimensional position information that is associated with time information of the time such images are captured. Specifically, the acquiring device 4 acquires the three-dimensional position information of the time each of the temporal-order ultrasound images are captured, in a manner associated with the time information at which such an image is captured. For example, the acquiring device 4 acquires the three-dimensional position information acquired at time “t1”, associates the time information “t1” to the three-dimensional position information thus acquired, and notifies the controller 18 of the information. For example, the acquiring device 4 acquires the three-dimensional position information acquired at time “t2”, associates the time information “t2” to the three-dimensional position information thus acquired, and notifies the controller 18 of the information.

The rendering processor 16 generates a plurality of temporal-order probe image groups, based on the temporal-order three-dimensional position information and the time information. Specifically, the rendering processor 16 generates a plurality of temporal-order probe image groups based on the three-dimensional position information and time information at which each of the temporal-order ultrasound images is captured. For example, the rendering processor 16 generates a “probe image group (t1)” for displaying “3DP(t1)” that is a stereoscopic image of the ultrasound probe 1 (hereinafter, mentioned as a 3D probe image) at time “t1” based on three-dimensional position information acquired at the time “t1”. For example, the rendering processor 16 generates a “probe image group (t2)” for displaying “3DP(t2)” that is a 3D probe image at time “t2” based on the three-dimensional position information acquired at the time “t2”.

The controller 18 controls to display each of a plurality of temporal-order probe image groups and each of a plurality of temporal-order ultrasound images being characterizing images onto the monitor 2.

For example, under the control of the controller 18, the image generator 15 generates a “synthesized image group (t1)” in which the “probe image group (t1)”, the B-mode image “F1(t1)”, and the rendering image “F2” of the mammary gland tissues are synthesized in a positional relationship based on the three-dimensional position information acquired at time “t1”. The image generator 15 also generates a “synthesized image group (t2)” in which the “probe image group (t2)”, the B-mode image “F1(t2)”, and the rendering image “F2” of the mammary gland tissues are synthesized in a positional relationship based on the three-dimensional position information acquired at time “t2.

The controller 18 displays the synthesized image groups generated by the image generator 15 onto the monitor 2. In this manner, as illustrated in FIG. 10, the monitor 2 displays the “3D probe image 3DP(t1)” and the B-mode image “F1(t1)” on the rendering image “F2” of the mammary gland tissues, and displays the “3D probe image 3DP(t2)” and the B-mode image “F1(t2)” on the rendering image “F2” of the mammary gland tissues. In the example of displaying images illustrated in FIG. 10, the 3D probe image and the B-mode image captured at each time is displayed in a manner superimposed over one another. However, the embodiment is also applicable to an example in which the stereoscopic image of the ultrasound probe 1 and the B-mode image captured at each time are displayed as a movie, or displayed in parallel.

Alternatively, a following process may be performed to generate the temporal-order probe image groups. Using the function of drawing two-dimensional images, the rendering processor 16 generates a plurality of body surface images that are a plurality of temporal-order abutting surface images, by changing the form of the body surface image that is the abutting surface image over time based on the three-dimensional position information and the time information. Specifically, using the function of drawing two-dimensional images, the rendering processor 16 generate the temporal-order body surface images by changing the form of the body surface image over time based on the three-dimensional position information and the time information at which each of the temporal-order ultrasound images is captured. The controller 18 controls to display each one of the temporal-order probe image groups and each one of the temporal-order body surface images being characterizing images onto the monitor 2.

For example, the rendering processor 16 generates a body mark representing how the body surface of the subject P is pressed over time, based on the three-dimensional position information acquired when elastography is conducted. The image generator 15 then generates a plurality of temporal-order synthesized image groups under the control of the controller 18; in each of the temporal-order synthesized image groups, each of the temporal-order probe image groups and each of a plurality of temporal-order body marks are synthesized in a positional relationship based on the three-dimensional position information acquired corresponding time.

The controller 18 displays the synthesized image groups generated by the image generator 15 onto the monitor 2. In this manner, the monitor 2 displays an stereoscopic image depicting how the body surface is pressed by an operation of the ultrasound probe 1, as illustrated in FIG. 11. Although not illustrated in FIG. 11, the controller 18 may also display an elastography generated by the image generator 15, along with the probe image group and the body marks.

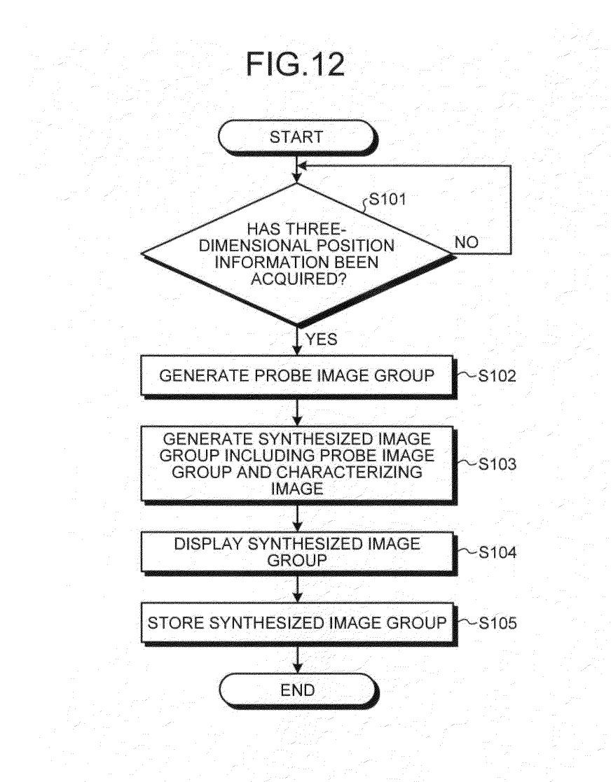

A process performed by the ultrasonic diagnostic apparatus according to the first embodiment will now be explained with reference to FIG. 12. FIG. 12 is a flowchart for explaining the process performed by the ultrasonic diagnostic apparatus according to the first embodiment. Explained below is a process performed after an ultrasound image is started being captured, holding the ultrasound probe 1 against the subject P.

As illustrated in FIG. 12, the controller 18 in the ultrasonic diagnostic apparatus according to the first embodiment determines if three-dimensional position information is acquired by the acquiring device 4 (Step S101). If three-dimensional position information has not been acquired (No at Step S101), the controller 18 waits until three-dimensional position information is acquired.

By contrast, if three-dimensional position information is acquired (Yes at Step S101), the rendering processor 16 generates a probe image group under the control of the controller 18 (Step S102). The image generator 15 also generates an ultrasound image in parallel with the probe image group.

The image generator 15 then generates a synthesized image group of the probe image group and the characterizing image under the control of the controller 18 (Step S103). The monitor 2 then displays the synthesized image group under the control of the controller 18 (Step S104).

The controller 18 then stores the synthesized image group in the image memory 17 (Step S105), and ends the process. When a plurality of synthesized image groups are generated in a temporal order, the controller 18 continues to perform the determining process at Step S101. When a deformed body mark being a characterizing image is to be displayed, the deformed body mark is generated by the rendering processor 16 at Step S102, along with the probe image group.

As described above, in the first embodiment, for example, by looking at the stereoscopic image illustrated in FIG. 8, an observer of the monitor 2 can recognize three-dimensionally what kind of operation conditions the ultrasound probe 1 was in when the B-mode image “F1” was captured. Furthermore, in the first embodiment, for example, by looking at the stereoscopic image illustrated in FIG. 9, an observer of the monitor 2 can recognize three-dimensionally in which direction and at what angle the ultrasound probe 1 was held against the body surface of the subject P when the B-mode image “F1” was captured. Furthermore, by requesting a synthesized image group stored in the image memory 17 and the like to be displayed, an observer of the monitor 2 can check a three-dimensional operation condition of the ultrasound probe 1 at the time the B-mode image “F1” was captured.

Furthermore, by looking at the stereoscopic image illustrated in FIG. 10, an observer of the monitor 2 can understand temporally how an operator operated the ultrasound probe 1 three-dimensionally while holding the ultrasound probe 1 against the body surface of the subject P, when the ultrasound images for image diagnosis were captured. Furthermore, by looking at the stereoscopic image illustrated in FIG. 11, an observer of the monitor 2 can understand how far the body surface of the subject P was pressed using the ultrasound probe 1 when the elastography generated by the image generator 15 was captured. Furthermore, by requesting a plurality of temporal-order synthesized image groups stored in the image memory 17 and the like to be displayed, an observer of the monitor 2 can check how the ultrasound probe 1 was operated three-dimensionally at the time such images were captured.

Therefore, according to the first embodiment, three-dimensional information related to an operation of the ultrasound probe 1 can be presented. Furthermore, use of the ultrasonic diagnostic apparatus according to the first embodiment can contribute to improvement in quality of information provided to radiologist reading an ultrasound image, improvement in reproducibility in re-examinations, and improvement in quality of diagnosis by reducing a variation caused by different examination skills of operators.

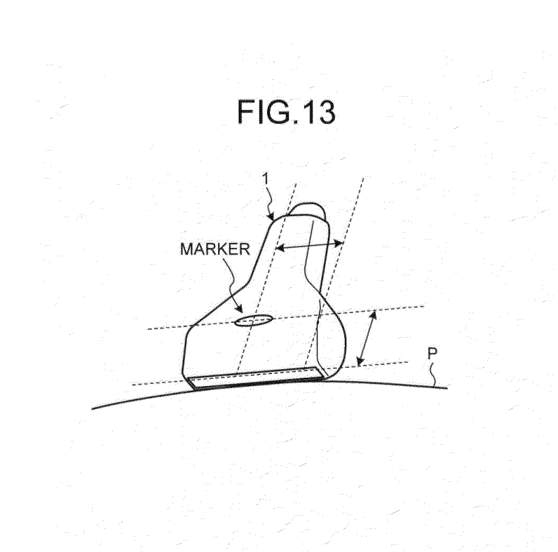

Explained above is an example in which the three-dimensional position information is acquired using magnetic sensors; however, means for acquiring the three-dimensional position information is not limited thereto. FIG. 13 is a schematic for explaining a variation of how the three-dimensional position information is acquired. For example, in this variation, a marker is attached on the surface of the ultrasound probe 1, as illustrated in FIG. 13. In such a configuration, a distance between the marker and the surface on which the transducer elements are arranged, a distance between the marker and an end of the ultrasound probe 1, and the like illustrated in FIG. 13 are stored in the internal storage 19 as offset information.

While images are being captured, a plurality of cameras are used to shoot the marker from a plurality of directions. The controller 18 then acquires the three-dimensional position information by analyzing a plurality of images thus shot using offset information, for example. Alternatively, the three-dimensional position information may also be acquired by an acceleration sensor.

Explained in a second embodiment is an example in which a probe image group is generated for an ultrasound image captured in the past.

For example, in the second embodiment, the controller 18 acquires the three-dimensional position information via the input device 3, instead of the acquiring device 4. In other words, the controller 18 acquires the three-dimensional position information of when an ultrasound image was captured based on input information input via the input device 3 by an observer who is looking at an ultrasound image generated by the image generator 15 in the past.

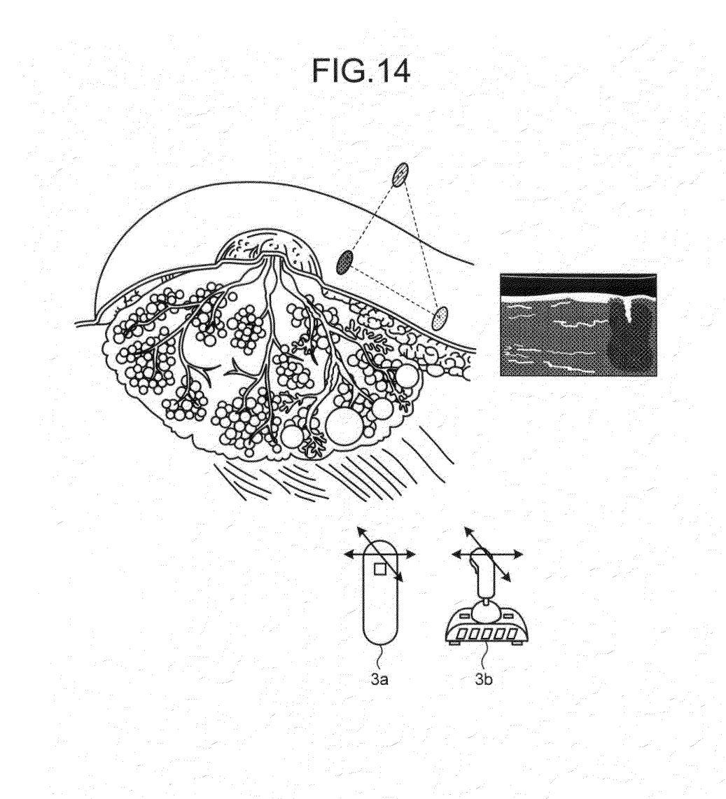

FIG. 14 is a schematic for explaining the second embodiment. To explain using an example, under the control of the controller 18, the monitor 2 displays a past ultrasound image (past image) designated by an observer, and a rendering image depicting a region captured in the past image designated by the observer, as illustrated in FIG. 14. The observer inputs a direction and an angle of the ultrasound probe 1 used when the operator himself/herself captured the ultrasound image in the past, by making operations on a haptic device 3a having an acceleration sensor or a joystick 3b provided to the input device 3, for example, while looking at the monitor 2. Using such input information, the controller 18 acquires the three-dimensional position information of when the past image was captured, and the rendering processor 16 generates a probe image group using the three-dimensional position information, which is based on input information.

In the manner described above, the controller 18 displays a stereoscopic image such as one illustrated in FIG. 9 onto the monitor 2. The controller 18 also stores a synthesized image group used in displaying the stereoscopic image such as one illustrated in FIG. 9 in the image memory 17, for example.

The input information related to the three-dimensional position information may be input using a mouse or a keyboard provided to the input device 3. Alternatively, the input information related to the three-dimensional position information may be acquired by the acquiring device 4 using the ultrasound probe 1 explained in the first embodiment on which the sensor group 41 is mounted as an input device.

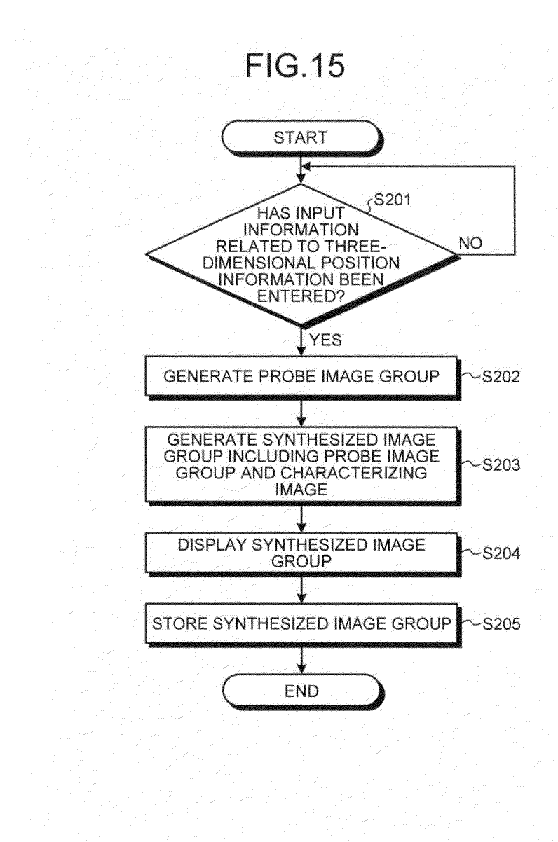

A process performed by the ultrasonic diagnostic apparatus according to the second embodiment will now be explained with reference to FIG. 15. FIG. 15 is a flowchart for explaining the process performed by the ultrasonic diagnostic apparatus according to the second embodiment. Explained below is a process performed after a past ultrasound image is displayed onto the monitor 2.

As illustrated in FIG. 15, the controller 18 in the ultrasonic diagnostic apparatus according to the second embodiment determines if input information related to the three-dimensional position information is entered by an observer of the monitor 2 via the input device 3 (Step S201). If input information related to the three-dimensional position information has not been entered (No at Step S201), the controller 18 waits until the information is entered.

By contrast, if input information related to the three-dimensional position information is entered (Yes at Step S201), the rendering processor 16 generates a probe image group under the control of the controller 18 (Step S202).

The image generator 15 then generates a synthesized image group including the probe image group and the characterizing image, under the control of the controller 18 (Step S203). The monitor 2 then displays the synthesized image group under the control of the controller 18 (Step S204).

The controller 18 then stores the synthesized image group in the image memory 17 (Step S205), and ends the process.

As described above, in the second embodiment, a probe image group can be synthesized and displayed based on input information received from an observer who is looking at an ultrasound image captured in the past. Therefore, in the second embodiment, three-dimensional information related to an operation of the ultrasound probe 1 can be presented for an ultrasound image captured in the past. The second embodiment is also applicable for allowing a plurality of temporal-order probe image groups to be generated, by looking at a plurality of temporal-order ultrasound images captured in the past.

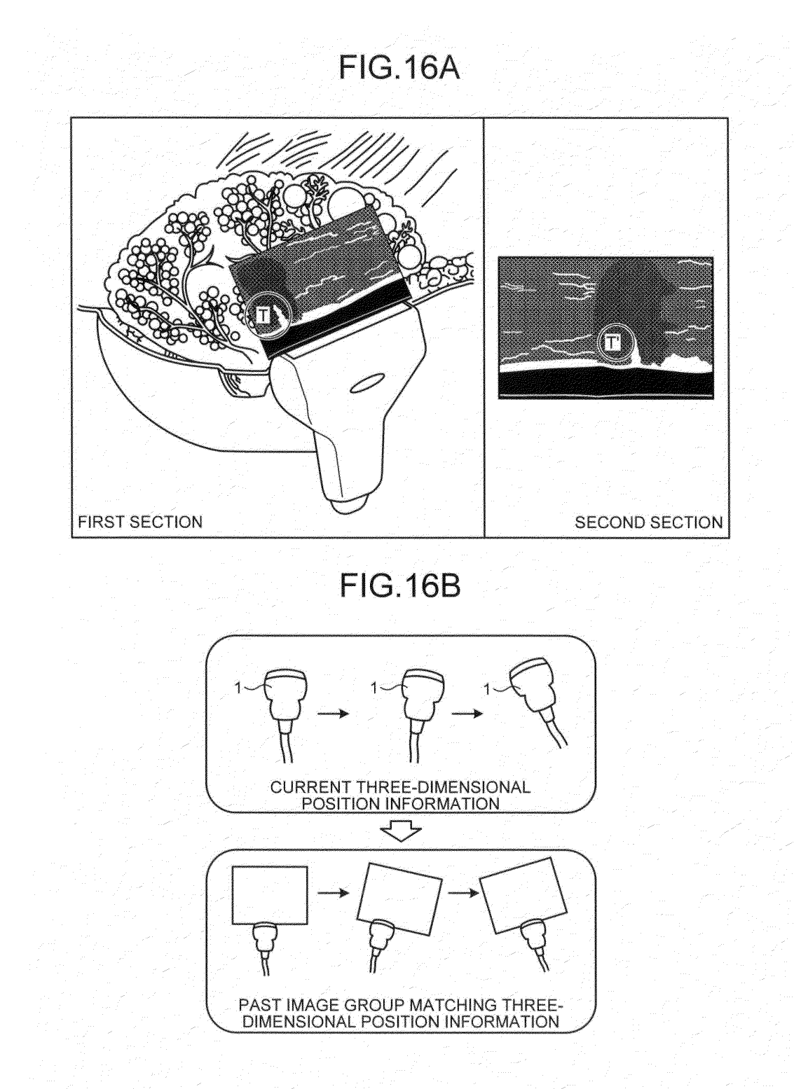

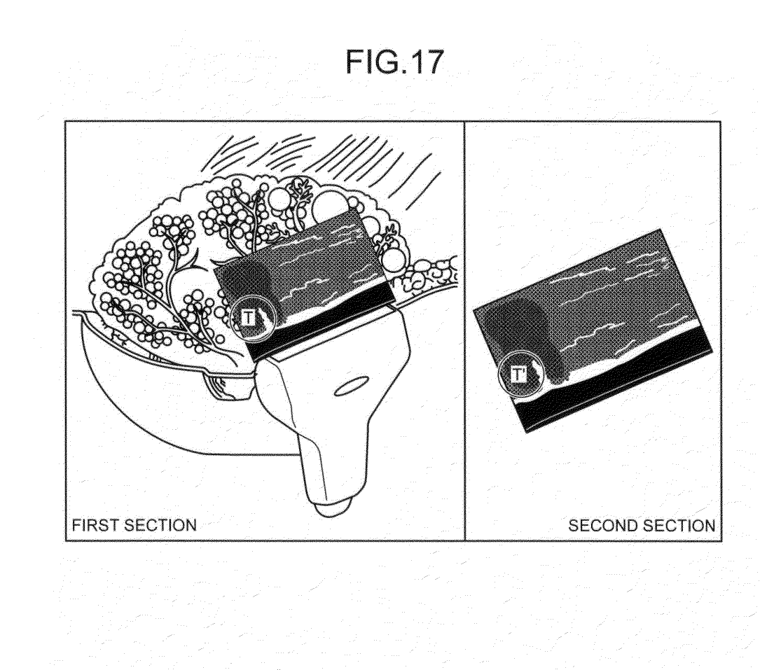

Explained in a third embodiment with reference to FIGS. 16A, 16B, 17, and the like is an example in which the synthesized image group explained in the first embodiment is used to capture an image of the same region as that captured in an ultrasound image of the past. FIGS. 16A, 16B, and 17 are schematics for explaining the third embodiment.

The controller 18 according to the third embodiment displays a past ultrasound image of the subject P and a probe image group acquired from the image memory 17 in a first section of a display area of the monitor 2. Specifically, when an operator designates a past examination ID of the subject P, the controller 18 acquires a synthesized image group having the examination ID thus designed from the image memory 17. Hereinafter, a past synthesized image group having a designated examination ID is referred to as a past image group.

For example, the past image group acquired by the controller 18 is “a plurality of temporal-order past image groups” including past B-mode images in the temporal order, a rendering image of a captured region, and probe image groups of the ultrasound probe 1 of when these past images were captured, such as the example illustrated in FIG. 10. In such a condition, the operator designates a past image group in which a past tumor region “T” that is a characterizing region requiring a follow-up observation is most clearly represented, while looking at a movie of the temporal-order past image groups. The monitor 2 then displays the past image group in which the past tumor region “T” is represented, in the first section illustrated in FIG. 16A.

The controller 18 according to the third embodiment then displays the ultrasound image of the subject P being currently captured in a second section of the display area of the monitor 2. Through such a display control, the operator of the ultrasound probe 1 being an observer of the monitor 2 displays a B-mode image including a current tumor region “T′” corresponding to the past tumor region “T” in the second section, by operating the ultrasound probe 1 on which the sensor group 41 are mounted (see FIG. 16A).

The controller 18 according to the third embodiment then controls to display the past ultrasound image and the probe image group matching the three-dimensional position information of the ultrasound probe 1 of when the current ultrasound image is captured, acquired by the acquiring device 4, in the first section. In other words, the acquiring device 4 acquires the three-dimensional position information of the ultrasound probe 1 of when the current ultrasound image (hereinafter, a current image) is captured, as illustrated in FIG. 16B. The controller 18 then selects a past image group in which a probe image group matching such three-dimensional position information is synthesized, among the “temporal-order past image groups”, and displays the past image group in the first section. In other words, the controller 18 displays a past image group matching the three-dimensional position information, as illustrated in FIG. 16B.

By looking at the images in the first section and the second section displayed under the display control described above, the operator of the ultrasound probe 1 keeps operating the ultrasound probe 1 until a current image in which a current tumor region “T′” is represented at approximately the same position as the position where the past tumor region “T” is displayed.

In this manner, the monitor 2 displays the current image in which the current tumor region “T′” is represented at the same position as the past tumor region “T” in the second section, as illustrated in FIG. 17. The current image displayed in the second section is caused to be stored in the image memory 17 by the controller 18, when the operator makes an OK input by pressing an OK button on the input device 3, for example.

Depending on an operation condition of the current ultrasound probe 1, a past image group that is synthesized with a probe image group matching the three-dimensional position information might not be selectable from the “temporal-order past image groups”. In such a case, the rendering processor 16 newly generates a probe image group matching the three-dimensional position information. The rendering processor 16 also generates an ultrasound image matching the three-dimensional position information by an interpolation.

For example, the controller 18 selects two past ultrasound images generated when past three-dimensional position information having coordinates closer to those of the current three-dimensional position information is acquired. The rendering processor 16 then newly generates an ultrasound image matching the current three-dimensional position information by an interpolation using the depth information of each of these two ultrasound images selected by the controller 18. In this manner, the image generator 15 newly generates a synthesized image group matching the current three-dimensional position information as a past image group.

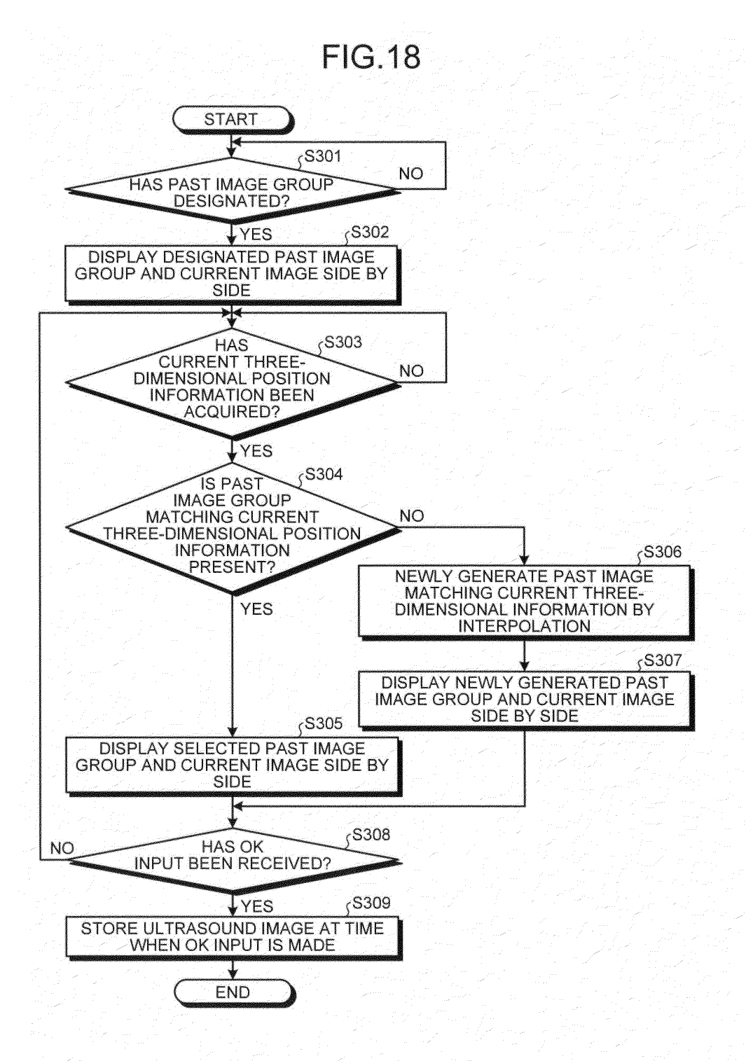

A process performed by the ultrasonic diagnostic apparatus according to the third embodiment will now be explained with reference to FIG. 18. FIG. 18 is a flowchart for explaining the process performed by the ultrasonic diagnostic apparatus according to the third embodiment. Explained below is a process performed after a plurality of temporal-order past image groups are displayed as a movie in the first section of the monitor 2.

As illustrated in FIG. 18, the controller 18 in the ultrasonic diagnostic apparatus according to the third embodiment determines if a past image group in which a characterizing region is most clearly represented is designated (Step S301). If a past image group has not been designated (No at Step S301), the controller 18 waits until a past image group is designated.

By contrast, if a past image group is designated (Yes at Step S301), the monitor 2 displays the past image group thus designated and a current image in parallel, under the control of the controller 18 (Step S302).

The controller 18 then determines if the acquiring device 4 has acquired the current three-dimensional position information (Step S303). If current three-dimensional position information has not been acquired (No at Step S303), the controller 18 waits until the current three-dimensional position information is acquired. By contrast, if the current three-dimensional position information is acquired (Yes at Step S303), the controller 18 determines if a past image group matching the current three-dimensional position information is present (Step S304).

If a past image group matching the current three-dimensional position information is present (Yes at Step S304), the controller 18 selects the matching past image group, and displays the past image group thus selected and the current image in parallel (Step S305).

By contrast, if no past image group matches the current three-dimensional position information (No at Step S304), the rendering processor 16 and the image generator 15 cooperate with each other to newly generate a past image group matching the current three-dimensional position information by an interpolation, under the control of the controller 18 (Step S306). The controller 18 then displays the newly generated past image group and the current image in parallel (Step S307).

Subsequent to Step S305 or Step S307, the controller 18 determines if an OK input is received from the operator (Step S308). If no OK input is received (No at Step S308), the controller 18 goes back to Step S303, and determines if the current three-dimensional position information is acquired.

By contrast, if an OK input is received (Yes at Step S308), the controller 18 stores the ultrasound image (current image) at the time such an OK input is made (Step S309), and ends the process.

As described above, in the third embodiment, when a follow-up observation is to be performed to a characterizing region in an ultrasound image captured in a past examination, the observer of the monitor 2 can observe an ultrasound image being currently captured while looking at a stereoscopic image of the ultrasound probe 1 matching the current three-dimensional position information and a past ultrasound image captured at the current three-dimensional position information. In other words, the observer of the monitor 2 can make a follow-up observation on the current characterizing region by operating the ultrasound probe 1 with an understanding of how the ultrasound probe 1 was three-dimensionally operated in the past. Therefore, in the third embodiment, the quality of reproducibility in re-examinations can be further improved.

Explained in the first to the third embodiments was an example in which the monitor 2 is a nine-parallax monitor. However, the first to the third embodiments are also applied in an example in which the monitor 2 is a two-parallax monitor.

Furthermore, explained in the first to the third embodiments is an example in which the ultrasound image synthesized to the probe image group is a B-mode image. However, the first to the third embodiments may represent an example in which the ultrasound image synthesized to the probe image group is a color Doppler image. Furthermore, the first to the third embodiments may also represent an example in which the ultrasound image synthesized to the probe image group is a parallax image group that is generated from three-dimensional ultrasound image data.

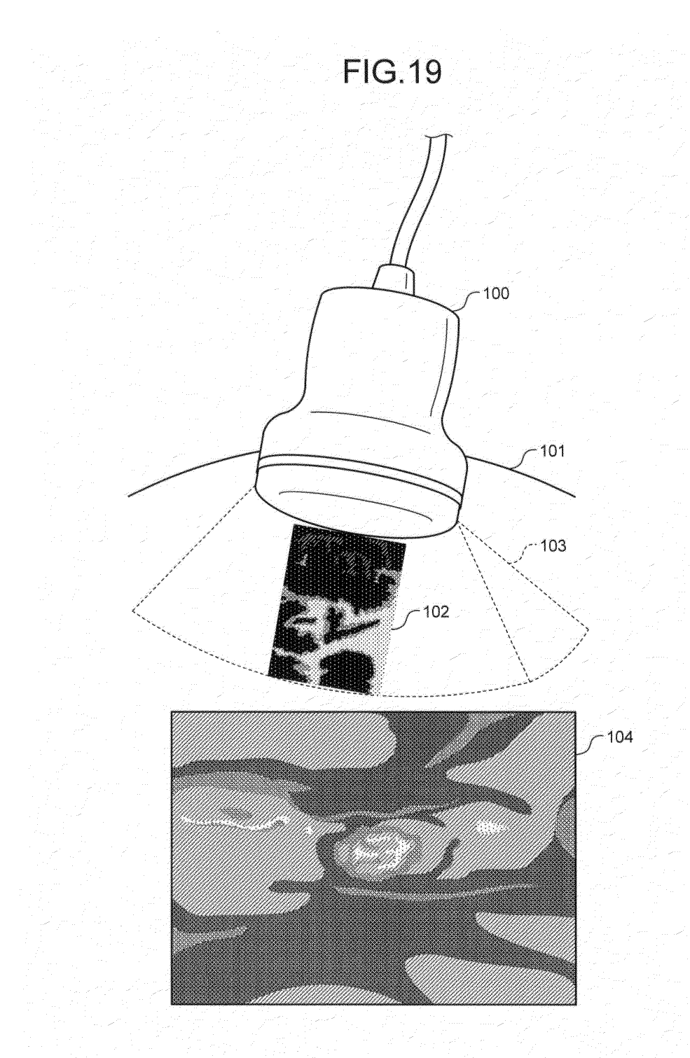

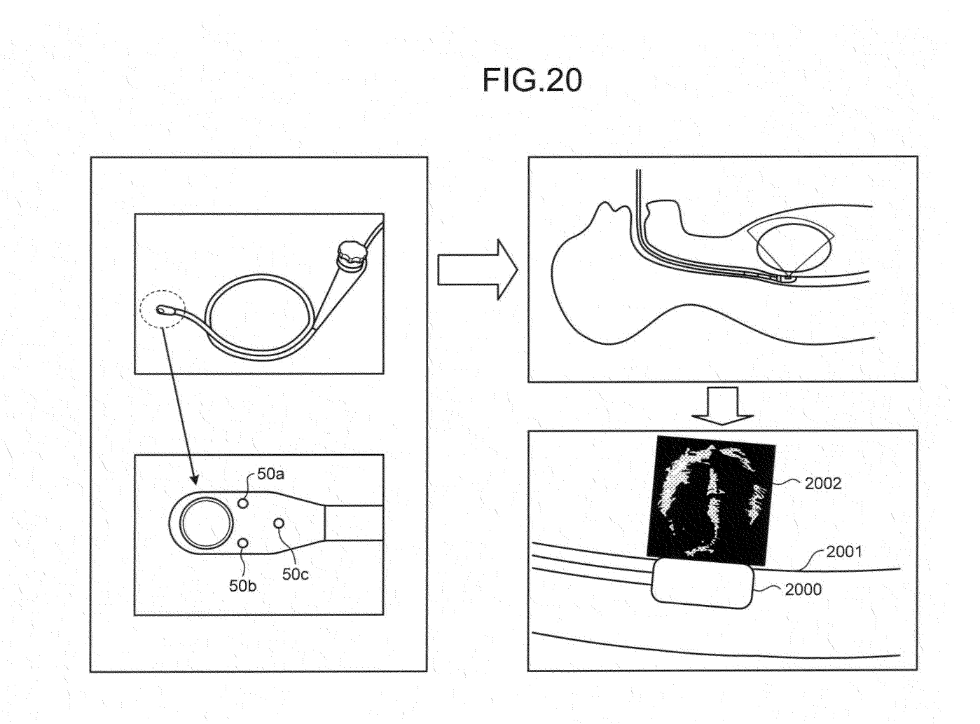

A variation of an ultrasound image synthesized to a probe image group will now be explained with reference to FIGS. 19 and 20. FIGS. 19 and 20 are schematics for explaining a variation of the first to the third embodiments.

Recently, a virtual endoscopic (VE) image that allows inside of a lumen to be observed is generated and displayed from a volume data including a lumen. As a possible way to display a VE image, a flythrough view, in which VE images are displayed as a movie by moving the viewpoint position along the centerline of the lumen, is known. When a flythrough view of a mammary gland is to be produced using an ultrasonic diagnostic apparatus, for example, an operator collects “volume data including the mammary gland” by holding an ultrasound probe 1 capable of three-dimensional scanning (e.g., a mechanical scanning probe) against the breast of the subject P. The rendering processor 16 illustrated in FIG. 1 extracts an area corresponding to the lumen from volume data by extracting pixels (voxels) with luminance corresponding to the luminance of the lumen, for example. The rendering processor 16 then applies a thinning process to the lumen area thus extracted, to extract the centerline of the lumen, for example. The rendering processor 16 generates a VE image from the viewpoint position along the centerline by perspective projection, for example. The rendering processor 16 generates a plurality of VE images for a flythrough view, by moving the viewpoint position along the centerline of the lumen.

When a flythrough view is to be provided, the acquiring device 4 acquires the three-dimensional position information of the ultrasound probe 1 of when volume data used in generating the VE images was collected. The controller 18 then causes the monitor 2 to display a synthesized image group including the characterizing image and each of the probe images included in the probe image group by performing the controlling process explained above in the first embodiment, and stores the synthesized image group in the image memory 17. FIG. 19 is an example of an image displayed on the monitor 2 when a flythrough view is provided under the control of the controller 18. An image 100 illustrated in FIG. 19 is a 3D probe image that an observer can observe the ultrasound probe 1 stereoscopically, being a result of displaying the probe image group generated by the rendering processor 16 based on the three-dimensional position information onto the monitor 2.

An image 101 illustrated in FIG. 19 is a body surface image as an abutting surface image, and is a 3D body mark that is a stereoscopic representation of the breast that is a captured region, for example. An image 102 illustrated in FIG. 19 is an image of an area including the lumen area used in providing a flythrough view, in the volume data. For example, the image 102 illustrated in FIG. 19 is a lumen image generated by the rendering processor 16 in a cavity mode, in which lower luminance values are reversed with higher luminance values. By reversing luminance values, the visibility of the lumen can be improved. An image 103 drawn in the dotted line in FIG. 19 is a schematic representation of a range of the three-dimensional ultrasound scan, generated by the rendering processor 16 based on the three-dimensional position information and conditions of the ultrasound scan. An image 104 illustrated in FIG. 19 is a VE image displayed in a flythrough view. The images 101 to 104 are characterizing images. In the example illustrated in FIG. 19, the images 100 to 103 are displayed onto the monitor 2 in a positional relationship based on the three-dimensional position information, under the control of the controller 18. In the example illustrated in FIG. 19, the image 104 is arranged below the images 100 to 103, under the control of the controller 18.

The controller 18 may arrange the image 103 instead of the image 102. Furthermore, the image 102 may be a volume rendering image generated by the rendering processor 16 using a single viewpoint position from the volume data. Furthermore, the image 102 may be a stereoscopic image that is nine-parallax images generated and displayed by the rendering processor 16 from the volume data using nine viewpoint positions. The image 100 may also be generated using information input by an observer, as explained in the second embodiment.

By looking at the stereoscopic image whose example is illustrated in FIG. 19, an observer can understand how the ultrasound probe 1 is three-dimensionally operated in order to produce a flythrough view of the image 104. Furthermore, by storing a synthesized image group generated for displaying a stereoscopic image illustrated in FIG. 19 and performing the process explained in the third embodiment, for example, a flythrough view can be performed using a VE image group at an approximately the same position as that in the image 104 provided with a flythrough view in the past examination.

The controlling process explained in the first to the third embodiments may also be applied to an example in which a luminal probe is used. The upper left diagram in FIG. 20 illustrates an example of a TEE probe. Magnetic sensors 50a, 50b, and 50c are mounted on the tip of the TEE probe, as illustrated in the lower left diagram in FIG. 20. The arrangement of the magnetic sensors 50a, 50b, and 50c illustrated in the lower left diagram in FIG. 20 is just an example. As long as three-dimensional position information of the TEE probe as the ultrasound probe 1 can be acquired, the magnetic sensors 50a, 50b, and 50c may be arranged in any positions.

An operator inserts the TEE probe into the esophagus of the subject P, as illustrated in the upper right diagram in FIG. 20, and performs two-dimensional scanning or three-dimensional scanning of a heart while holding the tip of the TEE probe against the inner wall of the esophagus. In such a condition, the acquiring device 4 acquires the three-dimensional position information of the TEE probe of when the data of an area including the heart is collected. The controller 18 then displays the synthesized image group including the characterizing image and each probe image in the probe image group onto the monitor 2 and stores the synthesized image group in the image memory 17 by performing the controlling process explained in the first embodiment. The lower right diagram in FIG. 20 illustrates an example of images displayed onto the monitor 2 under the control of the controller 18 while a transesophageal echocardiographic examination is conducted. An image 2000 illustrated in the lower right diagram in FIG. 20 is a 3D probe image that is achieved as a result of displaying a probe image group generated by the rendering processor 16 based on the three-dimensional position information onto the monitor 2, and that allows an observer to observe the TEE probe stereoscopically. An image 2001 illustrated in the lower right diagram in FIG. 20 is an abutting surface image, and is a body mark indicating the inner wall of the esophagus. As an abutting surface image, a 3D body mark being a stereoscopic representation of the heart, which is a captured region, or a surface rendering image of the heart may be used in addition to the image 2001. As an abutting surface image, for example, a human body model may also be used, as illustrated in the upper right diagram in FIG. 20. An image 2002 illustrated in the lower right diagram in FIG. 20 is an MPR image generated by the rendering processor 16 from the volume data including the heart. The image 2001 and the image 2002 are the characterizing images. In the example illustrated in the lower right diagram in FIG. 20, the images 2000 to 2002 are displayed onto the monitor 2 in a positional relationship based on the three-dimensional position information, under the control of the controller 18.

The image 2002 may also be a volume rendering image generated by the rendering processor 16 from volume data using a single viewpoint position. The image 2002 may also be a stereoscopic image achieved by displaying nine-parallax images generated by the rendering processor 16 from the volume data using nine viewpoint positions. The image 2000 may be generated from information input by an observer, as explained in the second embodiment.

By looking at images whose example is illustrated in the lower right diagram in FIG. 20, the observer can understand how the TEE probe was operated three-dimensionally in order to display the image 2002. Furthermore, by storing a synthesized image group generated for displaying a stereoscopic image illustrated in the lower right diagram in FIG. 20 and performing the process explained in the third embodiment, for example, it is possible to display an ultrasound image at approximately the same position as that in the image 2002 displayed in the past examination. The acquiring device 4 may also acquire information such as the length and the depth into which the TEE probe is inserted, using the positional relationship between the transmitter 42 and the subject P, for example. The controller 18 may add such information to the information of the synthesized image group. In this manner, an operation of the TEE probe required to collect the image 2002 can be presented more precisely.

As explained above, according to the first to the third embodiments and the variation thereof, three-dimensional information related to an operation of an ultrasound probe can be presented.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a display unit configured to display a stereoscopic image that is stereoscopically perceived by an observer, by displaying a parallax image group that is parallax images having a given parallax number;
an image generator configured to generate an ultrasound image based on reflection waves received by an ultrasound probe held against a subject;
an acquiring unit configured to acquire three-dimensional position information of the ultrasound probe of when an ultrasound image is captured;
a rendering processor configured to generate a probe image group that is a parallax image group for allowing the ultrasound probe to be virtually perceived as a stereoscopic image through a volume rendering process based on the three-dimensional position information acquired by the acquiring unit; and
a controller configured to control to display at least one of the ultrasound image and an abutting surface image depicting an abutting surface of the subject against which the ultrasound probe is held, as a characterizing image depicting a characteristic of a condition under which the ultrasound image is captured, and the probe image group onto the display unit in a positional relationship based on the three-dimensional position information.

2. The ultrasonic diagnostic apparatus according to claim 1, wherein
the image generator is configured to generate a plurality of ultrasound images in a temporal order based on reflection waves received by the ultrasound probe in a temporal order,
the acquiring unit is configured to acquire three-dimensional position information in the temporal order in association with time information at which the ultrasound images are captured,
the rendering processor is configured to generate a plurality of temporal-order probe image groups based on the three-dimensional position information and the time information, and
the controller is configured to control to display each one of the temporal-order probe image groups and each one of the temporal-order ultrasound images as the characterizing image onto the display unit.

3. The ultrasonic diagnostic apparatus according to claim 2, wherein
the rendering processor is configured to generate a plurality of temporal-order abutting surface images by changing a form of the abutting surface image in the temporal order based on the three-dimensional position information and the time information, and
the controller is configured to control to display each one of the temporal-order probe image groups and each one of the temporal-order abutting surface images as the characterizing image onto the display unit.

4. The ultrasonic diagnostic apparatus according to claim 1, wherein the acquiring unit is configured to acquire the three-dimensional position information using a position sensor mounted on the ultrasound probe.

5. The ultrasonic diagnostic apparatus according to claim 1, wherein
the acquiring unit is configured to acquire, based on input information input via a given input unit by an observer who is looking at an ultrasound image generated by the image generator in past, three-dimensional position information of when the ultrasound image is captured, and
the rendering processor is configured to generate the probe image group using three-dimensional position information based on the input information.

6. The ultrasonic diagnostic apparatus according to claim 1, wherein the controller is configured to store the probe image group and the characterizing image displayed onto the display unit in a given storage unit.

7. The ultrasonic diagnostic apparatus according to claim 6, wherein
the controller is configured to display a past ultrasound image of a subject and a probe image group acquired from the given storage unit in a first display area of the display unit, and to display an ultrasound image of the subject being currently captured in a second display area of the display unit, and
the controller is further configured to control to display a past ultrasound image and a probe image group matching three-dimensional position information, acquired by the acquiring unit, of the ultrasound probe of when a current ultrasound image is captured in the first display area.
